SEQUENCE LISTING (31 sequences)

SEQ ID NO: 1 - protein, 16 aa, synthetic construct
RSSQSLENSNAKTYLN

SEQ ID NO: 2 - protein, 7 aa, synthetic construct
GVSNRFS

SEQ ID NO: 3 - protein, 9 aa, synthetic construct
LQVTHVPYT

SEQ ID NO: 4 - protein, 10 aa, synthetic construct
GYTFTNYWIG

SEQ ID NO: 5 - protein, 17 aa, synthetic construct
DIYPGGDYTNYNEKFKG

SEQ ID NO: 6 - protein, 11 aa, synthetic construct
ARWGLGYYFDY

SEQ ID NO: 7 - protein, 16 aa, synthetic construct
RSSQSLENSNGKTYLN

SEQ ID NO: 8 - protein, 16 aa, synthetic construct
RSSQSLENTNGKTYLN

SEQ ID NO: 9 - protein, 112 aa, synthetic construct
DVVMTQSPLSLPVTLGQPASISCRSSQSLENSNAKTYLNWFQQRPGQSPRRLIYGVSNRFSGVPDRFSGSGSGTDFTLKISRVEAEDVGVYFCLQVTHVPYTFGQGTKLEIK

SEQ ID NO: 10 - protein, 112 aa, synthetic construct
DIVMTQTPLSLSVTPGQPASISCRSSQSLENSNAKTYLNWYLQKPGQSPQLLIYGVSNRFSGVPDRFSGSGSGTDFTLKISRVEAEDVGVYYCLQVTHVPYTFGQGTKVEIK

SEQ ID NO: 11 - protein, 118 aa, synthetic construct
QVQLVQSGAEVKKPGSSVKVSCKASGYTFTNYWIGWVRQAPGQGLEWVGDIYPGGDYTNYNEKFKGRVTITADKSTSTAYMELSSLRSEDTAVYYCARWGLGYYFDYWGQGTMVTVSS

SEQ ID NO: 12 - protein, 118 aa, synthetic construct
QVQLVQSGPEVKKPGESLKISCKGSGYTFTNYWIGWVRQAPGKGLEWMGDIYPGGDYTNYNEKFKGQVTISADKSISTAYLQWSSLKASDTAIYYCARWGLGYYFDYWGRGTLVTVSS

SEQ ID NO: 13 - protein, 219 aa, synthetic construct
DVVMTQSPLSLPVTLGQPASISCRSSQSLENSNAKTYLNWFQQRPGQSPRRLIYGVSNRFSGVPDRFSGSGSGTDFTLKISRVEAEDVGVYFCLQVTHVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 448 aa, synthetic construct
QVQLVQSGAEVKKPGSSVKVSCKASGYTFTNYWIGWVRQAPGQGLEWVGDIYPGGDYTNYNEKFKGRVTITADKSTSTAYMELSSLRSEDTAVYYCARWGLGYYFDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 219 aa, synthetic construct
DIVMTQTPLSLSVTPGQPASISCRSSQSLENSNAKTYLNWYLQKPGQSPQLLIYGVSNRFSGVPDRFSGSGSGTDFTLKISRVEAEDVGVYYCLQVTHVPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 448 aa, synthetic construct
QVQLVQSGPEVKKPGESLKISCKGSGYTFTNYWIGWVRQAPGKGLEWMGDIYPGGDYTNYNEKFKGQVTISADKSISTAYLQWSSLKASDTAIYYCARWGLGYYFDYWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 346 aa, Homo sapiens
MAMVSEFLKQAWFIENEEQEYVQTVKSSKGGPGSAVSPYPTFNPSSDVAALHKAIMVKGVDEATIIDILTKRNNAQRQQIKAAYLQETGKPLDETLKKALTGHLEEVVLALLKTPAQFDADELRAAMKGLGTDEDTLIEILASRTNKEIRDINRVYREELKRDLAKDITSDTSGDFRNALLSLAKGDRSEDFGVNEDLADSDARALYEAGERRKGTDVNVFNTILTTRSYPQLRRVFQKYTKYSKHDMNKVLDLELKGDIEKCLTAIVKCATSKPAFFAEKLHQAMKGVGTRHKALIRIMVSRSEIDMNDIKAFYQKMYGISLCQAILDETKGDYEKILVALCGGN

SEQ ID NO: 18 - protein, 204 aa, Homo sapiens
MNLILRYTFSKMAMVSEFLKQAWFIENEEQEYVQTVKSSKGGPGSAVSPYPTFNPSSDVAALHKAIMVKGVDEATIIDILTKRNNAQRQQIKAAYLQETGKPLDETLKKALTGHLEEVVLALLKTPAQFDADELRAAMKGLGTDEDTLIEILASRTNKEIRDINRVYREELKRDLAKDITSDTSGDFRNALLSLAKGDRSEDFG

SEQ ID NO: 19 - protein, 115 aa, Homo sapiens
MAMVSEFLKQAWFIENEEQEYVQTVKSSKGGPGSAVSPYPTFNPSSDVAALHKAIMVKGVDEATIIDILTKRNNAQRQQIKAAYLQETGKPLDETLKKALTGHLEEVVLALLKTP

SEQ ID NO: 20 - protein, 20 aa, synthetic construct
MVSSAQFLGLLLLCFQGTRC

SEQ ID NO: 21 - protein, 19 aa, synthetic construct
MGWTLVFLFLLSVTAGVHS

SEQ ID NO: 22 - protein, 239 aa, synthetic construct
MVSSAQFLGLLLLCFQGTRCDVVMTQSPLSLPVTLGQPASISCRSSQSLENSNAKTYLNWFQQRPGQSPRRLIYGVSNRFSGVPDRFSGSGSGTDFTLKISRVEAEDVGVYFCLQVTHVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 467 aa, synthetic construct
MGWTLVFLFLLSVTAGVHSQVQLVQSGAEVKKPGSSVKVSCKASGYTFTNYWIGWVRQAPGQGLEWVGDIYPGGDYTNYNEKFKGRVTITADKSTSTAYMELSSLRSEDTAVYYCARWGLGYYFDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 239 aa, synthetic construct
MVSSAQFLGLLLLCFQGTRCDIVMTQTPLSLSVTPGQPASISCRSSQSLENSNAKTYLNWYLQKPGQSPQLLIYGVSNRFSGVPDRFSGSGSGTDFTLKISRVEAEDVGVYYCLQVTHVPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 467 aa, synthetic construct
MGWTLVFLFLLSVTAGVHSQVQLVQSGPEVKKPGESLKISCKGSGYTFTNYWIGWVRQAPGKGLEWMGDIYPGGDYTNYNEKFKGQVTISADKSISTAYLQWSSLKASDTAIYYCARWGLGYYFDYWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - DNA, 720 bp, synthetic construct
  source 1..720: mol_type = other DNA
atggtgtcatccgctcaatttctcggtttgcttctcctgtgtttccaaggcacccgctgcgacgtggtcatgacccagagcccactgagccttccggtcaccttgggacagcccgcctcaatttcatgccggtccagccagtccctggagaactccaacgccaagacctatctgaactggttccagcagcgccctggacagtccccgaggcgcctgatctacggcgtcagcaacaggttctcgggcgtgccggacagattctccggctccggaagcggaactgacttcaccctgaaaatctcaagagtggaagccgaggacgtgggcgtgtacttctgcctccaagtcacgcacgtgccgtacactttcggacaagggactaagctggagatcaagcggaccgtggcggccccctctgtgttcattttccctccctcggacgaacagctgaagtcgggaacagcctccgtcgtgtgcctgctcaacaacttctacccccgggaagcgaaggtccagtggaaagtggataacgcactccaatcggggaactcccaggaatccgtgactgagcaggactcgaaggattccacttactccctgtcgtccaccctgactctgagcaaggccgactacgagaagcataaggtctacgcctgcgaagtgacccaccagggtctgagctcccctgtgaccaagagctttaatcggggcgaatgttga

SEQ ID NO: 27 - DNA, 1404 bp, synthetic construct
  source 1..1404: mol_type = other DNA
atgggatggactctcgtgttcctttttctcctctctgtcactgccggggtgcattcgcaagtccagctggtgcagtcgggagcagaggtgaaaaagcccggatcgtcagtgaaggtcagctgcaaagcctcgggatacactttcaccaactactggattggatgggtcagacaggcccccggccaaggactggagtgggtcggcgacatctaccctgggggcgactataccaactacaacgaaaagttcaagggacgcgtgacaattaccgccgataagagcaccagcactgcctacatggagcttagctcattgcggtccgaggataccgctgtgtactactgtgcgcggtggggccttggttactacttcgactactggggacagggtaccatggtcacggtgtcctccgcgtccaccaagggtccctccgtgttccctctcgcgccgtcctcaaagtctacctccggtggaactgccgcgctcggttgtctcgtgaaggactacttcccggagcctgtgactgtctcctggaactccggggccctcaccagcggagtgcacactttccccgccgtgctgcaatcctccggcctgtacagcctgtcctccgtcgtgactgtgcctagctcctccctgggaacccagacctacatctgcaacgtgaaccacaagccctccaacaccaaggtcgacaagaaggtcgaaccgaagtcgtgcgacaagactcatacgtgccctccttgcccggccccggaactgctgggaggcccatccgtgttcctgttcccacccaagcctaaggataccctgatgatcagcagaacaccggaagtgacctgtgtggtggtggacgtcagccacgaagatcccgaggtcaagttcaattggtacgtggacggggtggaggtgcacaacgcaaagaccaagccccgggaggaacagtacaactccacctatcgcgtggtgtcggtgctgacggtgctgcaccaggactggttgaacggaaaggagtataagtgcaaagtgtcgaacaaggccctgcccgctcctatcgaaaagaccatctccaaggccaagggccagccgcgggaaccccaggtctacactctcccaccgagccgcgacgaactgactaagaatcaagtgtcgctgacttgcctcgtcaagggcttctacccgtccgacatcgccgtggaatgggagagcaacggccagccggaaaacaactacaagaccacccctcccgtgctggattccgacgggtccttcttcctgtactcaaaactgaccgtggataagtccagatggcagcagggcaatgtcttttcatgctccgtgatgcacgaggctctgcataaccactacacccagaagtcgctgtccctgtccccggggaagtga

SEQ ID NO: 28 - DNA, 720 bp, synthetic construct
  source 1..720: mol_type = other DNA
atggtgtcatccgctcaatttctcggtttgcttctcctgtgtttccaaggcacccgctgcgacatcgtcatgacccagaccccattgagcctttccgtcacgccgggacagcccgcctccatttcctgccgctcaagccagtccctggagaactcaaacgccaagacctacctgaattggtatctgcagaagcctggacagagcccgcagctgctgatctacggcgtcagcaacaggttctcgggcgtgccggacagattctccggctccggaagcggaactgacttcaccctgaaaatctcacgcgtggaagccgaggacgtgggcgtgtactactgcctccaagtcacccacgtgccgtacactttcggacaagggactaaggtcgagatcaagcggaccgtggcggccccctctgtgttcattttccctccctcggacgaacagctgaagtcgggaacagcctccgtcgtgtgcctgctcaacaacttctacccccgggaagcgaaggtccagtggaaagtggataacgcactccaatcggggaactcccaggaatccgtgactgagcaggactcgaaggattccacttactccctgtcgtccaccctgactctgagcaaggccgactacgagaagcataaggtctacgcctgcgaagtgacccaccagggtctgagctcccctgtgaccaagagctttaatcggggcgaatgttga

SEQ ID NO: 29 - DNA, 1404 bp, synthetic construct
  source 1..1404: mol_type = other DNA
atgggatggactctcgtgttcctttttctcctctctgtcactgccggggtgcattcgcaagtccagctggtgcagtcgggaccagaggtgaaaaagcccggagagtcacttaagatcagctgcaaaggctcgggatacactttcaccaactactggattggttgggtcagacaggcccccggcaaaggactggagtggatgggcgacatctaccctgggggcgactataccaactacaacgaaaagttcaagggacaagtgacaatttcggccgataagagcattagcactgcataccttcagtggagctcattgaaggcctccgataccgctatctactactgtgcgcggtggggcctgggatactacttcgactactggggaaggggtaccttggtcacggtgtcctccgcgtccaccaagggtccctccgtgttccctctcgcgccgtcctcaaagtctacctccggtggaactgccgcgctcggttgtctcgtgaaggactacttcccggagcctgtgactgtctcctggaactccggggccctcaccagcggagtgcacactttccccgccgtgctgcaatcctccggcctgtacagcctgtcctccgtcgtgactgtgcctagctcctccctgggaacccagacctacatctgcaacgtgaaccacaagccctccaacaccaaggtcgacaagaaggtcgaaccgaagtcgtgcgacaagactcatacgtgccctccttgcccggccccggaactgctgggaggcccatccgtgttcctgttcccacccaagcctaaggataccctgatgatcagcagaacaccggaagtgacctgtgtggtggtggacgtcagccacgaagatcccgaggtcaagttcaattggtacgtggacggggtggaggtgcacaacgcaaagaccaagccccgggaggaacagtacaactccacctatcgcgtggtgtcggtgctgacggtgctgcaccaggactggttgaacggaaaggagtataagtgcaaagtgtcgaacaaggccctgcccgctcctatcgaaaagaccatctccaaggccaagggccagccgcgggaaccccaggtctacactctcccaccgagccgcgacgaactgactaagaatcaagtgtcgctgacttgcctcgtcaagggcttctacccgtccgacatcgccgtggaatgggagagcaacggccagccggaaaacaactacaagaccacccctcccgtgctggattccgacgggtccttcttcctgtactcaaaactgaccgtggataagtccagatggcagcagggcaatgtcttttcatgctccgtgatgcacgaggctctgcataaccactacacccagaagtcgctgtccctgtccccggggaagtga

SEQ ID NO: 30 - protein, 219 aa, Mus musculus
DAVMTQTPLSLPVSLGDQVSISCRSSQSLENSNGKTYLNWYLQKPGQSPQLLIYGVSNRFSGVLDRFSGSGSGTDFTLKISRVEAEDLGVYFCLQVTHVPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 31 - protein, 454 aa, Mus musculus
QVQLQQSGPELVRPGTSVKMSCKASGYTFTNYWIGWAKQRPGHGLEWIGDIYPGGDYTNYNEKFKGKATLTADKSSSTAYMQFSSLTSEDSAIYYCARWGLGYYFDYWGQGITLTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

(19) United States
(12) Patent Application Publication
(10) Pub. No.: US 2025/0025553 A1
(43) Pub. Date: Jan. 23, 2025

(54) COMBINATION THERAPY FOR CANCER

(71) Applicant: Medannex Ltd., Edinburgh, Lothian (GB)

(72) Inventors: Henry Charles Wilson HAYS, Edinburgh, Lothian (GB); Chirstopher Barry WOOD, Edinburgh, Lothian (GB); Fiona Carolyn DEMPSEY, Edinburgh, Lothian (GB); Scott James CRICHTON, Edinburgh, Lothaian (GB); James Alexander INGHAM, Edinburgh, Lothian (GB); Charlene FABIAN, Edinburgh, Lothian (GB)

(73) Assignee: Medannex Ltd., Edinburgh, Lothian (GB)

(21) Appl. No.: 18/710,903

(22) PCT Filed: Nov. 18, 2022
(86) PCT No.: PCT/EP2022/082515

§ 371 Date: May 16, 2024

(30) Foreign Application Priority Data
Nov. 18, 2021 (GB) 2116680.6
Jun. 16, 2022 (GB) 2208893.4

(51) Int. Cl. A61K 39/395; A61K 31/337; A61K 31/513; A61K 31/69; A61K 31/7068; A61K 33/243; A61K 38/07; A61P 35/00; C07K 16/18

(52) U.S. Cl. CPC A61K 39/3955; A61K 31/337; A61K 31/513; A61K 31/69; A61K 31/7068; A61K 33/243; A61K 38/07; A61P 35/00; C07K 16/18; C07K 2317/24; C07K 2317/622

(57) ABSTRACT

The present invention relates to the use of a specific binding molecules which binds human ANXA1 in combination with a second active agent for use in the treatment of cancer. Second active agents include thy midylate synthetase inhibitors, nucleobase analogues, checkpoint inhibitors, proteasome inhibitors, taxanes, platinum-based chemotherapy agents and nucleoside analogues. Preferred cancers for treatment are pancreatic cancer, colorectal cancer, breast cancer, lung cancer, myeloma and mantle cell lymphoma. Related kits, products and uses are also provided.

### FIELD

The present invention relates to the treatment of cancer with a specific binding molecule against annexin-A1 (ANXA1) in combination with certain other therapeutic agents.

### BACKGROUND

Cancer is a group of diseases characterised by abnormal cell growth. Characteristically, the abnormal cell growth associated with cancer results in the formation of a tumour (a solid mass of cells formed due to abnormal cell growth), though this is not always the case (particularly in cancers of the blood). In 2010 across the world more people (about 8 million) died from cancer than any other single cause (Lozano et al., Lancet 380:2095-2128, 2012). Furthermore, as populations across the world age, cancer rates are expected to increase. There is thus an urgent need for new and improved therapies for cancer.

Moreover, many cancer deaths are a result of a cancer becoming resistant to chemotherapy drugs. Methods by which cancers become drug-resistant are reviewed in Housman et al. (Cancers 6:1769-1792, 2014). As detailed therein, cancers may become drug-resistant by a variety of different mechanisms, including inactivation or metabolism of drugs (or the prevention of their metabolic activation), mutation or alteration of drug target and drug efflux via ABC transporters. Such mechanisms can result in cancers becoming multidrug resistant (MDR). The development of resistance to drug-based therapies is a significant challenge in oncology today. New treatment options for cancers that are, or have become, resistant to traditional chemotherapeutics are therefore needed.

The present invention provides new therapeutic options for cancer, specifically new therapies in which a specific binding molecule (such as an antibody) against annexin-A1 (ANXA1) is used in combination with certain specific partner drugs. As shown in the Examples below, the combinations provided herein are particularly effective in treating cancer or certain types of cancer.

Full length human ANXA1 has the amino acid sequence set forth in SEQ ID NO: 17. ANXA1 is a member of the annexin protein family. Most proteins of this family, including ANXA1, are characterised by the presence of a “core” region comprising four homologous, repeating domains, each of which comprises at least one Ca²⁺-binding site. Each member of the family is distinguished by a unique N-terminal region. ANXA1 is a monomeric amphipathic protein, predominantly located in the cytoplasm of cells in which it is expressed. However, ANXA1 can also be exported, resulting in cell surface localisation (D'Acquisto et al., Br. J. Pharmacol. 155:152-169, 2008).

ANXA1 is known to play a role in regulation of the immune system, being involved in the homeostasis of various cell types of both the innate and adaptive immune systems. For instance, ANXA1 has been shown to exert homeostatic control over cells of the innate immune system such as neutrophils and macrophages, and also to play a role in T cells by modulating the strength of T cell receptor (TCR) signalling (D'Acquisto et al., Blood 109:1095-1102, 2007). Use of a neutralising antibody against ANXA1 to inhibit its roles in the adaptive immune system has been shown to be effective in the treatment of various T cell-mediated diseases, including autoimmune diseases such as rheumatoid arthritis and multiple sclerosis (WO 2010/064012; WO 2011/154705).

Antibodies against ANXA1 have also been shown to be useful in the treatment of certain psychiatric conditions, in particular anxiety, obsessive-compulsive disorder (OCD) and related diseases (WO 2013/088111), though the mechanism by which this occurs is unknown.

A number of monoclonal antibodies that recognise human ANXA1 are disclosed in WO 2018/146230. As detailed in WO 2020/030827, these antibodies were found to bind human ANXA1 at a discontinuous epitope comprising the amino acids at positions 197-206, 220-224 and 227-237 (i.e. at an epitope comprising the amino acids at positions 197-206, 220-224 and 227-237 of SEQ ID NO: 17). The antibodies disclosed in WO 2018/146230 have particularly advantageous properties, in that they are able to bind to human ANXA1 with very high affinity. The antibodies disclosed in WO 2018/146230 were subsequently shown to possess a potent anti-cancer activity (WO 2020/030827). As demonstrated in WO 2020/030827, the antibodies demonstrated an anti-proliferative effect against multiple cancer cell lines, and also demonstrated therapeutic efficacy in a murine model of triple-negative breast cancer.

The present inventors have now discovered that combining treatment with the anti-ANXA1 antibodies disclosed in WO 2018/146230 with certain other specific therapeutic agents provides an unexpectedly enhanced anti-cancer effect.

### SUMMARY OF INVENTION

Thus, in a first aspect the invention provides a specific binding molecule which binds human ANXA1 and a second active agent for use in the treatment of cancer in a subject, wherein:
(i) the specific binding molecule comprises the complementarity-determining regions (CDRs) VLCDR1, VLCDR2, VLCDR3, VHCDR1, VHCDR2 and VHCDR3, each of said CDRs having an amino acid sequence as follows:
VLCDR1 has the sequence set forth in SEQ ID NO: 1, 7 or 8, or a modified version thereof comprising a conservative amino acid substitution at position 9 and/or 11;
VLCDR2 has the sequence set forth in SEQ ID NO: 2;
VLCDR3 has the sequence set forth in SEQ ID NO: 3;
VHCDR1 has the sequence set forth in SEQ ID NO: 4;
VHCDR2 has the sequence set forth in SEQ ID NO: 5; and
VHCDR3 has the sequence set forth in SEQ ID NO: 6; and
(ii) the second active agent is selected from a thymidylate synthetase inhibitor, a nucleobase analogue, a checkpoint inhibitor which blocks the interaction between PD-1 and PD-L1 and a proteasome inhibitor. Alternatively expressed, the invention provides the specific binding molecule as defined herein for use in the treatment of cancer in a subject, wherein in said treatment said specific binding molecule and a second active agent as defined herein is to be administered to said subject, i.e. said specific binding molecule and said second active agent is used in combination in said treatment.

In a second aspect the invention provides a specific binding molecule which binds human ANXA1 and a second active agent for use in the treatment of breast cancer in a subject, wherein the specific binding molecule is as defined above in respect of the first aspect, and the second active agent is selected from a taxane and a platinum-based chemotherapy agent. Alternatively expressed, the invention provides the specific binding molecule as defined herein for use in the treatment of breast cancer in a subject, wherein in said treatment said specific binding molecule and a second active agent as defined herein is to be administered to said subject, i.e. said specific binding molecule and said second active agent is used in combination in said treatment.

In a third aspect the invention provides a specific binding molecule which binds human ANXA1 and a second active agent for use in the treatment of pancreatic cancer in a subject, wherein the specific binding molecule is as defined above in respect of the first aspect, and the second active agent is a nucleoside analogue. Alternatively expressed, the invention provides the specific binding molecule as defined herein for use in the treatment of pancreatic cancer in a subject, wherein in said treatment said specific binding molecule and a second active agent as defined herein is to be administered to said subject, i.e. said specific binding molecule and said second active agent is used in combination in said treatment.

Relatedly, in a fourth aspect the invention provides a method of treating cancer in a subject, comprising administering to the subject a specific binding molecule which binds human ANXA1 and a second active agent, wherein the specific binding molecule is as defined above in respect of the first aspect and the second active agent is selected from a thymidylate synthetase inhibitor, a nucleobase analogue, a checkpoint inhibitor which blocks the interaction between PD-1 and PD-L1 and a proteasome inhibitor.

In a fifth aspect the invention provides a method of treating breast cancer in a subject, comprising administering to the subject a specific binding molecule which binds human ANXA1 and a second active agent, wherein the specific binding molecule is as defined above in respect of the first aspect and the second active agent is selected from a taxane and a platinum-based chemotherapy agent.

In a sixth aspect the invention provides a method of treating pancreatic cancer in a subject, comprising administering to the subject a specific binding molecule which binds human ANXA1 and a nucleoside analogue, wherein the specific binding molecule is as defined above in respect of the first aspect.

Relatedly, in a seventh aspect the invention provides the use of a specific binding molecule which binds human ANXA1 in the manufacture of a medicament for treating cancer, wherein the specific binding molecule is as defined above in respect of the first aspect, and said treatment of cancer comprises administering said medicament and a second active agent to a subject, wherein the second active agent is selected from a thymidylate synthetase inhibitor, a nucleobase analogue, a checkpoint inhibitor which blocks the interaction between PD-1 and PD-L1 and a proteasome inhibitor.

In an eighth aspect the invention provides the use of a specific binding molecule which binds human ANXA1 in the manufacture of a medicament for treating breast cancer, wherein the specific binding molecule is as defined above in respect of the first aspect, and said treatment of breast cancer comprises administering said medicament and a second active agent to a subject, wherein the second active agent is selected from a taxane and a platinum-based chemotherapy agent.

In a ninth aspect the invention provides the use of a specific binding molecule which binds human ANXA1 in the manufacture of a medicament for treating pancreatic cancer, wherein the specific binding molecule is as defined above in respect of the first aspect, and said treatment of pancreatic cancer comprises administering said medicament and a nucleoside analogue to the subject.

In a tenth aspect the invention provides a pharmaceutical composition comprising a specific binding molecule which binds human ANXA1, a second active agent and one or more pharmaceutically-acceptable diluents, carriers or excipients,
wherein the specific binding molecule and the second active agent are as defined above in respect of the first aspect.

In an eleventh aspect the invention provides a kit comprising a specific binding molecule which binds human ANXA1 and a second active agent, wherein the specific binding molecule and the second active agent are as defined above in respect of the first aspect.

In a twelfth aspect the invention provides a product comprising a specific binding molecule which binds human ANXA1 as defined above in respect of the first aspect and a second active agent for separate, simultaneous or sequential use in the treatment of cancer in a subject, wherein the second active agent is selected from a thymidylate synthetase inhibitor, a nucleobase analogue, a checkpoint inhibitor which blocks the interaction between PD-1 and PD-L1 and a proteasome inhibitor.

In a thirteenth aspect the invention provides a product comprising a specific binding molecule which binds human ANXA1 as defined above in respect of the first aspect and a second active agent for separate, simultaneous or sequential use in the treatment of breast cancer in a subject, wherein the second active agent is selected from a taxane and a platinum-based chemotherapy agent.

In a fourteenth aspect the invention provides a product comprising a specific binding molecule which binds human ANXA1 as defined above in respect of the first aspect and a nucleoside analogue for separate, simultaneous or sequential use in the treatment of pancreatic cancer in a subject.

As described hereinafter a third active agent may also be used in the above aspects of the invention.

### DESCRIPTION OF INVENTION

The invention provides new combinations which are effective in treating cancer. The combinations may serve to increase the efficacy of the components relative to their use separately. In one example, one of the components may potentiate the effects of an otherwise less than effective drug. This can be particularly useful to treat drug-resistant cancers, e.g. to provide a new treatment or to enhance efficacy of the drug to which the cancer has become resistant. Furthermore, in light of the enhanced effects achieved using the combinations, the invention allows lower levels of the components (e.g. the second or third active agent) to be used. In a preferred aspect the combination shows synergy, i.e. shows better than additive effects. Particularly in such cases it is possible to reduce the amount of one or both of the components (e.g. the second or third active agent) to be used and to potentiate the effect of a component (e.g. the second or third active agent) by its use in the combination. The specific binding molecule may provide the component of the combination that potentiates the activity of the second (or third) active agent, or vice versa.

As mentioned above, the invention provides (in part) a specific binding molecule which binds human ANXA1 for use in the treatment of cancer (or of certain particular cancers) in a subject. A “specific binding molecule” as defined herein is a molecule that binds specifically to a particular molecular partner, in this case human ANXA1. A molecule that binds specifically to human ANXA1 is a molecule that binds to human ANXA 1 with a greater affinity than that with which it binds to other molecules, or at least most other molecules. Thus, for example, if a specific binding molecule that binds human ANXA1 were contacted with a lysate of human cells, the specific binding molecule would bind primarily to ANXA1. In particular, the specific binding molecule binds to a sequence or configuration present on said human ANXA1. When the specific binding molecule is an antibody the sequence or configuration is the epitope to which the specific binding molecule binds. The ANXA1 epitope bound by the specific binding molecules for use according to the invention is detailed above.

The specific binding molecule for use herein does not necessarily bind only to human ANXA1: the specific binding molecule may cross-react with certain other undefined target molecules, or may display a level of non-specific binding when contacted with a mixture of a large number of molecules (such as a cell lysate or suchlike). For instance, the specific binding molecule may display a level of cross-reactivity with other members of the human annexin family, and/or with ANXA1 proteins from other animals. Regardless, a specific binding molecule for use according to the invention shows specificity for ANXA1. The skilled person will easily be able to identify whether a specific binding molecule shows specificity for ANXA1 using standard techniques in the art, e.g. ELISA, Western-blot, surface plasmon resonance (SPR), etc. In particular embodiments, the specific binding molecule for use herein binds human ANXA1 with a K_{D} (dissociation constant) of less than 20 nM, 15 nM or 10 nM. In a preferred embodiment, the specific binding molecule for use herein binds human ANXA1 with a K_{D} of less than 5 nM.

The K_{D} of the binding of the specific binding molecule to ANXA1 is preferably measured under binding conditions in which Ca²⁺ ions are present at a concentration of at least 1 mM, and optionally HEPES is present at a concentration of from 10-20 mM, and the pH is between 7 and 8, preferably of a physiological level between 7.2 and 7.5 inclusive. NaCl may be present, e.g. at a concentration of from 100-250 mM, and a low concentration of a detergent, e.g. polysorbate 20, may also be present. Such a low concentration may be e.g. from 0.01 to 0.5% v/v. A number of methods by which the K_{D} of an interaction between a specific binding molecule and its ligand may be calculated are well known in the art. Known techniques include SPR (e.g. Biacore) and polarization-modulated oblique-incidence reflectivity difference (OI-RD).

As described above, a molecule that “binds to human ANXA1” shows specificity for a human ANXA1 molecule. There are three human isoforms of human ANXA1, obtained from translation of four alternatively-spliced ANXA1 mRNAs. The full-length human ANXA1 protein is obtained from translation of the ANXA1-002 or ANXA1-003 transcript, and as noted above has the amino acid sequence set forth in SEQ ID NO: 17. The ANXA 1-004 and ANXA 1-006 transcripts encode fragments of the full-length human ANXA1 protein, which respectively have the amino acid sequences set forth in SEQ ID NOs: 18 and 19.

The specific binding molecule for use according to the invention binds to full-length human ANXA1 (i.e. ANXA1 of SEQ ID NO: 17, encoded by the ANXA1-002 or ANXA 1-003 transcript, which is a 346 amino acid protein). The specific binding molecule may also bind to particular fragments, parts or variants of full-length ANXA1, such as the fragments encoded by the ANXA1-004 and ANXA1-006 transcripts.

As discussed hereinafter, antibodies (and molecules containing CDRs) form preferred specific binding molecules for use according to the invention.

As mentioned above, a number of monoclonal antibodies that recognise human ANXA1 are disclosed in WO 2018/146230. One antibody disclosed in WO 2018/146230 has the following CDR sequences:

VLCDR1:
(SEQ ID NO: 1)
RSSQSLENSNAKTYLN;
VLCDR2:
(SEQ ID NO: 2)
GVSNRFS;
VLCDR3:
(SEQ ID NO: 3)
LQVTHVPYT;
VHCDR1:
(SEQ ID NO: 4)
GYTFTNYWIG;
VHCDR2:
(SEQ ID NO: 5)
and
DIYPGGDYTNYNEKFKG;
VHCDR3:
(SEQ ID NO: 6)
ARWGLGYYFDY.

Another antibody disclosed in WO 2018/146230 has the following CDR sequences:

VLCDR1:
(SEQ ID NO: 7)
RSSQSLENSNGKTYLN;
VLCDR2:
(SEQ ID NO: 2)
GVSNRFS;
VLCDR3:
(SEQ ID NO: 3)
LQVTHVPYT;
VHCDR1:
(SEQ ID NO: 4)
GYTFTNYWIG;
VHCDR2:
(SEQ ID NO: 5)
DIYPGGDYTNYNEKFKG;
and
VHCDR3:
(SEQ ID NO: 6)
ARWGLGYYFDY.

Another antibody disclosed in WO 2018/146230 has the following CDR sequences:

VLCDR1:
(SEQ ID NO: 8)
RSSQSLENTNGKTYLN;
VLCDR2:
(SEQ ID NO: 2)
GVSNRFS;
VLCDR3:
(SEQ ID NO: 3)
LQVTHVPYT;
VHCDR1:
(SEQ ID NO: 4)
GYTFTNYWIG;
VHCDR2:
(SEQ ID NO: 5)
DIYPGGDYTNYNEKFKG;
and
VHCDR3:
(SEQ ID NO: 6)
ARWGLGYYFDY.

(In line with standard nomenclature, VLCDR1, VLCDR2 and VLCDR3 respectively denote CDRs 1, 2 and 3 of the antibody light chain, while VHCDR1, VHCDR2 and VHCDR3respectively denote CDRs 1, 2 and 3 of the antibody heavy chain.)

Thus the antibodies disclosed in WO 2018/146230 have identical CDR sequences, save for the VLCDR1 sequences. The VLCDR1 sequence of SEQ ID NO: 7 is a wild-type VLCDR1 sequence, found in the murine antibody MDX-001 which was constructed from a minor mRNA sequence obtained from the hybridoma deposited with the ECACC having accession number 10060301.

Humanised versions of MDX-001 were generated and, surprisingly, modification of the VLCDR1 sequence in these humanised antibodies was found to yield enhanced antibodies. Substitution of the glycine residue at position 11 of SEQ ID NO: 7 enhances antibody stability and function. Without being bound by theory it is believed that this is achieved by removing a site for post-translational modification of the CDR. Specifically, it is believed that substitution of this glycine residue removes a deamidation site from the protein. The VLCDR1 sequence set forth in SEQ ID NO: 7 comprises the sequence motif Ser-Asn-Gly. This sequence motif is associated with deamidation of the Asn residue, which leads to conversion of the asparagine residue to aspartic acid or isoaspartic acid, which can affect antibody stability and target binding. Substitution of any one of the residues within the Ser-Asn-Gly motif is believed to remove the deamidation site.

As detailed in WO 2018/146230, antibodies in which the glycine residue at position 11 of SEQ ID NO: 7 (which is the glycine residue located within the above-described deamidation site) is substituted for alanine display enhanced binding to their target (ANXA1) relative to the native, MDX-001 antibody. The VLCDR1 comprising the substitution of glycine at position 11 for alanine has the amino acid sequence RSSQSLENSNAKTYLN (the residue in bold is the alanine introduced by the aforementioned substitution), which is set forth in SEQ ID NO: 1. Further, humanised antibodies comprising a VLCDR1 modified at position 9, by substitution of serine for threonine, were also found to display enhanced binding of ANXA1 relative to MDX-001. The VLCDR1 comprising the substitution of serine at position 9 for threonine has the amino acid sequence RSSQSLENTNGKTYLN (the residue in bold is the threonine introduced by the aforementioned substitution), which is set forth in SEQ ID NO: 8.

The specific binding molecule for use according to the present invention comprises the CDR sequences of any of the three antibodies disclosed in WO 2018/146230, or certain variants thereof. In particular, as noted above, VLCDR1 of the antibodies disclosed in WO 2018/146230 have been found to at least tolerate conservative amino acid substitutions at positions 9 and 11 of VLCDR1. Accordingly, the specific binding molecule for use according to the invention comprises CDRs having amino acid sequences as follows:
VLCDR1 has the sequence set forth in SEQ ID NO: 1, 7 or 8, or a modified version thereof comprising a conservative amino acid substitution at position 9 and/or 11;
VLCDR2 has the sequence set forth in SEQ ID NO: 2;
VLCDR3 has the sequence set forth in SEQ ID NO: 3;
VHCDR1 has the sequence set forth in SEQ ID NO: 4;
VHCDR2 has the sequence set forth in SEQ ID NO: 5; and
VHCDR3 has the sequence set forth in SEQ ID NO: 6.

The term “conservative amino acid substitution”, as used herein, refers to an amino acid substitution in which one amino acid residue is replaced with another amino acid residue having a similar side chain. Amino acids with similar side chains tend to have similar properties, and thus a conservative substitution of an amino acid important for the structure or function of a polypeptide may be expected to affect polypeptide structure/function less than a non-conservative amino acid substitution at the same position. Families of amino acid residues having similar side chains have been defined in the art, including basic side chains (e.g. lysine, arginine, histidine), acidic side chains (e.g. aspartic acid, glutamic acid), uncharged polar side chains (e.g. asparagine, glutamine, serine, threonine, tyrosine), non-polar side chains (e.g. glycine, cysteine, alanine, valine, leucine, isoleucine, proline, phenylalanine, methionine, tryptophan) and aromatic side chains (e.g. tyrosine, phenylalanine, tryptophan, histidine). Thus a conservative amino acid substitution may be considered to be a substitution in which a particular amino acid residue is substituted for a different amino acid in the same family.

Thus in a particular embodiment the specific binding molecule for use according to the invention comprises a VLCDR1 which is a modified version of SEQ ID NO: 1, 7 or 8 comprising a conservative amino acid substitution at position 9 relative to the sequence set forth in SEQ ID NO: 1, 7 or 8. In another embodiment the specific binding molecule for use according to the invention comprises a VLCDR1 which is a modified version of SEQ ID NO: 1, 7 or 8 comprising a conservative amino acid substitution at position 11 relative to the sequence set forth in SEQ ID NO: 1, 7 or 8. In another embodiment the specific binding molecule for use according to the invention comprises a VLCDR1 which is a modified version of SEQ ID NO: 1, 7 or 8 comprising conservative amino acid substitutions at both positions 9 and 11 relative to SEQ ID NO: 1, 7 or 8.

In a preferred aspect,
a) the conservative amino acid substitution at position 9 relative to the sequence set forth in SEQ ID NO: 7 or 1 (when the amino acid at that position is serine) is asparagine, glutamine, threonine or tyrosine;
b) the conservative amino acid substitution at position 9 relative to the sequence set forth in SEQ ID NO: 8 (when the amino acid at that position is threonine) is asparagine, glutamine, serine or tyrosine;
c) the conservative amino acid substitution at position 11 relative to the sequence set forth in SEQ ID NO: 7 or 8 (when the amino acid at that position is glycine) is cysteine, alanine, valine, leucine, isoleucine, proline, phenylalanine, methionine or tryptophan; or
d) the conservative amino acid substitution at position 11 relative to the sequence set forth in SEQ ID NO: 1 (when the amino acid at that position is alanine) is glycine, cysteine, valine, leucine, isoleucine, proline, phenylalanine, methionine or tryptophan.

In a preferred embodiment, the specific binding molecule for use according to the invention comprises CDRs having amino acid sequences as follows:
VLCDR1 has the sequence set forth in SEQ ID NO: 1, 7 or 8;
VLCDR2 has the sequence set forth in SEQ ID NO: 2;
VLCDR3 has the sequence set forth in SEQ ID NO: 3;
VHCDR1 has the sequence set forth in SEQ ID NO: 4;
VHCDR2 has the sequence set forth in SEQ ID NO: 5; and
VHCDR3 has the sequence set forth in SEQ ID NO: 6.

Most preferably, the specific binding molecule for use according to the invention comprises CDRs having amino acid sequences as follows:
VLCDR1 has the sequence set forth in SEQ ID NO: 1;
VLCDR2 has the sequence set forth in SEQ ID NO: 2;
VLCDR3 has the sequence set forth in SEQ ID NO: 3;
VHCDR1 has the sequence set forth in SEQ ID NO: 4;
VHCDR2 has the sequence set forth in SEQ ID NO: 5; and
VHCDR3 has the sequence set forth in SEQ ID NO: 6.

As indicated, the specific binding molecule for use according to the invention comprises 6 CDRs consisting of polypeptide sequences. As used herein, “protein” and “polypeptide” are interchangeable, and each refers to a sequence of 2 or more amino acids joined by one or more peptide bonds. Thus, the specific binding molecule may be a polypeptide. Alternatively, the specific binding molecule may comprise one or more polypeptides that comprise the CDR sequences. Preferably, the specific binding molecule for use according to the invention is an antibody or an antibody fragment.

The amino acids making up the sequence of the CDRs may include amino acids which do not occur naturally, but which are modifications of amino acids which occur naturally. Providing these non-naturally occurring amino acids do not alter the sequence and do not affect specificity, they may be used to generate CDRs described herein without reducing sequence identity, i.e. are considered to provide an amino acid of the CDR. For example derivatives of amino acids such as methylated amino acids may be used. In one embodiment the specific binding molecule for use according to the invention is not a natural molecule, i.e. is not a molecule found in nature.

The specific binding molecule for use according to the invention may be synthesised by any method known in the art. In particular, the specific binding molecule may be synthesised using a protein expression system, such as a cellular expression system using prokaryotic (e.g. bacterial) cells or eukaryotic (e.g. yeast, fungus, insect or mammalian) cells. An alternative protein expression system is a cell-free, in vitro expression system, in which a nucleotide sequence encoding the specific binding molecule is transcribed into mRNA, and the mRNA translated into a protein, in vitro. Cell-free expression system kits are widely available, and can be purchased from e.g. Thermo Fisher Scientific (USA). Alternatively, specific binding molecules may be chemically synthesised in a non-biological system. Liquid-phase synthesis or solid-phase synthesis may be used to generate polypeptides that may form or be comprised within the specific binding molecule for use according to the invention. The skilled person can readily produce specific binding molecules using appropriate methodology common in the art. In particular, the specific binding molecule may be recombinantly expressed in mammalian cells, such as CHO cells.

The specific binding molecule for use according to the invention may, if necessary, be isolated (i.e. purified). “Isolated”, as used herein, means that the specific binding molecule is the primary component (i.e. majority component) of any solution or suchlike in which it is provided. In particular, if the specific binding molecule is initially produced in a mixture or mixed solution, isolation of the specific binding molecule means that it has been separated or purified therefrom. Thus, for instance, if the specific binding molecule is a polypeptide, and said polypeptide is produced using a protein expression system as discussed above, the specific binding molecule is isolated such that it is the most abundant polypeptide in the solution or composition in which it is present, preferably constituting the majority of polypeptides in the solution or composition, and is enriched relative to other polypeptides and biomolecules present in the native production medium. In particular, the specific binding molecule for use according to the invention is isolated such that it is the predominant (majority) specific binding molecule in the solution or composition. In a preferred feature, the specific binding molecule is present in the solution or composition at a purity of at least 60, 70, 80, 90, 95 or 99% w/w when assessed relative to the presence of other components, particularly other polypeptide components, in the solution or composition.

If the specific binding molecule is a protein, e.g. produced in a protein expression system, a solution of the specific binding molecule may be analysed by quantitative proteomics to identify whether the specific binding molecule for use according to the invention is predominant and thus isolated. For instance, 2D gel electrophoresis and/or mass spectrometry may be used. Such isolated molecules may be present in preparations or compositions as described hereinafter.

The specific binding molecule of the present invention may be isolated using any technique known in the art. For instance, the specific binding molecule may be produced with an affinity tag such as a polyhistidine tag, a strep tag, a FLAG tag, an HA tag or suchlike, to enable isolation of the molecule by affinity chromatography using an appropriate binding partner, e.g. a molecule carrying a polyhistidine tag may be purified using Ni²⁺ ions. In embodiments in which the specific binding molecule is an antibody, the specific binding molecule may be isolated using affinity chromatography using one or more antibody-binding proteins, such as Protein G, Protein A, Protein A/G or Protein L. Alternatively, the specific binding molecule may be isolated by e.g. size-exclusion chromatography or ion-exchange chromatography. A specific binding molecule produced by chemical synthesis (i.e. by a non-biological method), by contrast, is likely to be produced in an isolated form. Thus, no specific purification or isolation step is required for a specific binding molecule for use according to the invention to be considered isolated, if it is synthesised in a manner that produces an isolated molecule.

Modifications to the amino acid sequences of the CDRs set out in SEQ ID NOs: 1-8 may be made using any suitable technique, such as site-directed mutagenesis of the encoding DNA sequence or solid state synthesis.

Specific binding molecules for use according to the invention may comprise, in addition to the above-described CDRs, linker moieties or framework sequences to allow appropriate presentation of the CDRs. Additional sequences may also be present which may conveniently confer additional properties, e.g. peptide sequences which allow isolation or identification of the molecules containing the CDRs such as those described hereinbefore. In such cases a fusion protein may be generated.

As stated above, the specific binding molecule for use according to the invention is preferably an antibody or an antibody fragment. The term “antibody” as used herein refers to antibodies containing all the features of a native immunoglobulin (as known in the art and see for example the description in WO 2020/030827, incorporated herein by reference) as well as variants of naturally occurring antibodies (or comprising all the features of a native immunoglobulin) that retain the CDRs but are presented in a different framework, as discussed hereinafter and which function in the same way, i.e. retain specificity for the antigen. Thus antibodies include functional equivalents or homologues in which naturally occurring domains have been replaced in part or in full with natural or non-natural equivalents or homologues which function in the same way.

When the specific binding molecule for use according to the invention is an antibody, it is preferably a monoclonal antibody. By “monoclonal antibody” is meant an antibody preparation consisting of a single antibody species, i.e. all antibodies in the preparation have the same amino acid sequences, including the same CDRs, and thus bind the same epitope on their target antigen (by “target antigen” is meant the antigen containing the epitope bound by a particular antibody, i.e. the target antigen of an anti-Anx-A1 antibody is Anx-A1) with the same effect. In other words, the antibody for use according to the invention is preferably not part of a polyclonal mix of antibodies.

In an antibody, as is well known in the art, the CDR sequences are located in the variable domains of the heavy and light chains. The CDR sequences sit within a polypeptide framework, which positions the CDRs appropriately for antigen binding. Thus the remainder of the variable domains (i.e. the parts of the variable domain sequences which do not form a part of any one of the CDRs) constitute framework regions. The N-terminus of a mature variable domain forms framework region 1 (FR1); the polypeptide sequence between CDR1 and CDR2 forms FR2; the polypeptide sequence between CDR2 and CDR3 forms FR3; and the polypeptide sequence linking CDR3 to the constant domain forms FR4. In an antibody or fragment thereof for use according to the invention the variable region framework regions may have any appropriate amino acid sequence such that the antibody or fragment thereof binds to human ANXA1 via its CDRs. The constant regions may be the constant regions of any mammalian (preferably human) antibody isotype.

In certain embodiments of the invention the specific binding molecule may be multi-specific, e.g. a bi-specific monoclonal antibody. A multi-specific binding molecule contains regions or domains (antigen-binding regions) that bind to at least two different molecular binding partners, e.g. bind to two or more different antigens or epitopes. In the case of a bi-specific antibody, the antibody comprises two heavy and light chains, in standard formation, except that the variable domains of the two heavy chains and the two light chains, respectively, are different, and thus form two different antigen-binding regions. In a multi-specific (e.g. bi-specific) binding molecule, e.g. monoclonal antibody, for use according to the invention, one of the antigen-binding regions has the CDR sequences of a specific binding molecule for use according to the invention as defined herein, and thus binds ANXA1. The other antigen-binding region(s) of the multi-specific binding molecule for use according to the invention are different to the antigen-binding regions formed by CDRs for use according to the invention, e.g. have CDRs with sequences different to those defined herein for the specific binding molecule for use according to the invention. The additional (e.g. second) antigen-binding region(s) of the specific binding molecule, e.g. in the bi-specific antibody, may also bind ANXA1, but at a different epitope to the first antigen-binding region which binds to ANXA1 (which has the CDRs of the specific binding molecule for use according to the invention). Alternatively, the additional (e.g. second) antigen-binding region(s) may bind additional (e.g. a second), different antigen(s) which is (are) not ANXA1. In an alternative embodiment, the two or more antigen-binding regions in the specific binding molecule, e.g. in an antibody, may each bind to the same antigen, i.e. provide a multivalent (e.g. bivalent) molecule.

The specific binding molecule may be an antibody fragment or synthetic construct capable of binding human ANXA1. Thus an antibody fragment for use according to the invention comprises an antigen-binding domain (i.e. the antigen-binding domain of the antibody from which it is derived), i.e. is antigen-binding fragment of an antibody. Antibody fragments are discussed in Rodrigo et al., Antibodies, Vol. 4(3), p. 259-277, 2015. Antibody fragments for use according to the invention are preferably monoclonal (i.e. they are not part of a polyclonal mix of antibody fragments). Antibody fragments include, for example, Fab, F(ab′)₂, Fab′ and Fv fragments. Fab fragments are discussed in Roitt et al, Immunology second edition (1989), Churchill Livingstone, London. A Fab fragment consists of the antigen-binding domain of an antibody, i.e. an individual antibody may be seen to contain two Fab fragments, each consisting of a light chain and its conjoined N-terminal section of the heavy chain. Thus a Fab fragment contains an entire light chain and the V_{H} and C_{H}¹ domains of the heavy chain to which it is bound. Fab fragments may be obtained by digesting an antibody with papain.

F(ab′)₂ fragments consist of the two Fab fragments of an antibody, plus the hinge regions of the heavy domains, including the disulphide bonds linking the two heavy chains together. In other words, a F(ab′)₂ fragment can be seen as two covalently joined Fab fragments. F(ab′)₂ fragments may be obtained by digesting an antibody with pepsin. Reduction of F(ab′)₂ fragments yields two Fab′ fragments, which can be seen as Fab fragments containing an additional sulfhydryl group which can be useful for conjugation of the fragment to other molecules.

Fv fragments consist of just the variable domains of the light and heavy chains. These are not covalently linked and are held together only weakly by non-covalent interactions. Fv fragments can be modified to produce a synthetic construct known as a single chain Fv (scFv) molecule. Such a modification is typically performed recombinantly, by engineering the antibody gene to produce a fusion protein in which a single polypeptide comprises both the V_{H} and V_{L} domains. scFv fragments generally include a peptide linker covalently joining the V_{H} and V_{L} regions, which contributes to the stability of the molecule. The linker may comprise from 1 to 20 amino acids, such as for example 1, 2, 3 or 4 amino acids, 5, 10 or 15 amino acids, or other intermediate numbers in the range 1 to 20 as convenient. The peptide linker may be formed from any generally convenient amino acid residues, such as glycine and/or serine. One example of a suitable linker is Gly₄Ser. Multimers of such linkers may be used, such as for example a dimer, a trimer, a tetramer or a pentamer, e.g. (Gly₄Ser)₂, (Gly₄Ser)₃, (Gly₄Ser)₄ or (Gly₄Ser)₅. However, it is not essential that a linker be present, and the V_{L} domain may be linked to the V_{H} domain by a peptide bond. An scFv is herein defined as an antibody fragment.

The specific binding molecule may be an analogue of an scFv. For example, the scFv may be linked to other specific binding molecules (for example other scFvs, Fab antibody fragments and chimeric IgG antibodies (e.g. with human frameworks)). The scFv may be linked to other scFvs so as to form a multimer that is a multi-specific binding protein, for example a dimer, a trimer or a tetramer. Bi-specific scFvs are sometimes referred to as diabodies, tri-specific scFvs as triabodies and tetra-specific scFvs as tetrabodies. In other embodiments the scFv for use according to the invention may be bound to other, identical scFv molecules, thus forming a multimer which is mono-specific but multi-valent, e.g. a bivalent dimer or a trivalent trimer may be formed.

Synthetic constructs that can be used include CDR peptides. These are synthetic peptides comprising antigen-binding determinants. Peptide mimetics can also be used. These molecules are usually conformationally-restricted organic rings that mimic the structure of a CDR loop and that include antigen-interactive side chains.

As noted above, the specific binding molecule for use according to the present invention comprises CDRs having the amino acid sequences set forth in SEQ ID NO: 1, 7 or 8 (or a variant thereof) and 2-6. As detailed, these are derived or modified from the murine antibody MDX-001. However, an antibody or fragment thereof for use according to the present invention is preferably humanised.

The antibody or antibody fragment for use according to the invention may be a human/mouse chimeric antibody, or preferably may be humanised. This is particularly the case for monoclonal antibodies and antibody fragments. Humanised or chimeric antibodies or antibody fragments are desirable when the molecule is to be used as a human therapeutic. Therapeutic treatment of humans with non-human (e.g. murine) antibodies can be ineffective for a number of reasons, e.g. a short in vivo half-life of the antibody; weak effector functions mediated by the foreign heavy chain constant region, due to low recognition of non-human heavy chain constant regions by Fc receptors on human immune effector cells; patient sensitisation to the antibody, and (in the context of murine antibodies) generation of a human anti-mouse antibody (HAMA) response; and neutralisation of the mouse antibody by HAMA leading to loss of therapeutic efficacy.

A chimeric antibody is an antibody with variable regions derived from one species and constant regions derived from another. Thus an antibody or antibody fragment for use according to the invention may be a chimeric antibody or chimeric antibody fragment, comprising murine variable domains and human constant domains.

An antibody for use according to the invention, including a chimeric antibody, may have the constant regions of any antibody isotype (in particular any human antibody isotype), and any sub-class within each isotype. For instance, the antibody may be of the isotype IgA, IgD, IgE, IgG or IgM antibody (i.e. the chimeric antibody may comprise the constant domains of heavy chains α, δ, ε, γ, or μ, respectively), though preferably the antibody for use according to the invention is of the IgG isotype. The light chain of the antibody (e.g. chimeric antibody) for use according to the invention may be either a κ or λ light chain, in particular it may comprise the constant region of a human λ light chain or a human κ light chain. A chimeric antibody fragment is, correspondingly, an antibody fragment comprising constant domains (e.g. an Fab, Fab′ or F(ab′)₂ fragment). The constant domains of a chimeric antibody fragment for use according to the invention may be as described above for a chimeric monoclonal antibody.

Chimeric antibodies may be generated using any suitable technique, e.g. recombinant DNA technology in which the DNA sequence of the murine variable domain is fused to the DNA sequence of the human constant domain(s) so as to encode a chimeric antibody. A chimeric antibody fragment may be obtained either by using recombinant DNA technology to produce a DNA sequence encoding such a polypeptide, or by processing a chimeric antibody for use according to the invention to produce the desired fragments, as described above. Chimeric antibodies can be expected to overcome the problems of a short in vivo half-life and weak effector functions associated with using a foreign, e.g. murine, antibody in human therapy, and may reduce the probability of patient sensitisation and HAMA occurring. However, patient sensitisation and HAMA may still occur when a chimeric antibody is administered to a human patient, due to the presence of murine sequences in the variable domains.

Preferably the antibody or antibody fragment for use according to the invention is therefore fully humanised. A humanised antibody is an antibody derived from another species, e.g. a mouse, in which the constant domains of the antibody chains are replaced with human constant domains, and the amino acid sequences of the variable regions are modified to replace the foreign (e.g. murine) framework sequences with human framework sequences, such that, preferably, the only non-human sequences in the antibody are the CDR sequences. A humanised antibody can overcome all the problems associated with therapeutic use of a non-human antibody in a human, including avoiding or minimising the probability of patient sensitisation and HAMA occurring.

Antibody humanisation is generally performed by a process known as CDR grafting, though any other technique in the art may be used. Antibody grafting is well described in Williams, D. G. et al., Antibody Engineering Vol. 1, edited by R. Kontermann and S. Dübel, Chapter 21, pp. 319-339. In this process, a chimeric antibody as described above is first generated. Thus in the context of humanisation of an antibody, the non-human constant domain is first replaced with a human constant domain, yielding a chimeric antibody comprising a human constant domain and non-human variable domain.

Subsequent humanisation of the foreign, e.g. murine, variable domains involves intercalating the murine CDRs from each immunoglobulin chain within the FRs of the most appropriate human variable region. This is done by aligning the murine variable domains with databases of known human variable domains (e.g. IMGT or Kabat). Appropriate human framework regions are identified from the best-aligned variable domains, e.g. domains with high sequence identity between the human and murine framework regions, domains containing CDRs of the same length, domains having the most similar structures (based on homology modelling), etc. The murine CDR sequences are then grafted into the lead human framework sequences at the appropriate locations using recombinant DNA technology, and the humanised antibodies then produced and tested for binding to the target antigen. The process of antibody humanisation is known and understood by the skilled individual, who can perform the technique without further instruction. Antibody humanisation services are also offered by a number of commercial companies, e.g. GenScript (USA/China) or MRC Technology (UK). Humanised antibody fragments can be easily obtained from humanised antibodies, as described above.

Thus the antibody or antibody fragment for use according to the invention may be derived from any species, e.g. it may be a murine antibody or antibody fragment. It is preferred, however, that the antibody or antibody fragment is a chimeric antibody or antibody fragment, i.e. that only the variable domains of the antibody or antibody fragment are non-human, and the constant domains are all human. Optimally, the antibody or antibody fragment for use according to the invention is a humanised antibody or antibody fragment.

Humanised versions of MDX-001 have been developed by the inventors, as detailed in WO 2018/146230. Humanised light chain variable domains have been developed with the amino acid sequences set forth in SEQ ID NO: 9 (known as the L1M2 variable region) and SEQ ID NO: 10 (known as the L2M2 variable region), containing the CDRs as described hereinbefore. In a particular embodiment, the antibody or fragment thereof for use according to the invention comprises a light chain variable region comprising or consisting of the amino acid sequence set forth in SEQ ID NO: 9 or SEQ ID NO: 10, or an amino acid sequence having at least 70% (preferably at least 80, 90, 95, 96, 97, 98 or 99%) sequence identity thereto, and in which the CDR sequences VLCDR1-3 are as defined above.

Humanised heavy chain variable domains have been developed with the amino acid sequences set forth in SEQ ID NO: 11 (known as the H4 variable region) and SEQ ID NO: 12 (known as the H2 variable region). In a particular embodiment, the antibody or fragment thereof for use according to the invention comprises a heavy chain variable region comprising or consisting of the amino acid sequence set forth in SEQ ID NO: 11 or SEQ ID NO: 12, or an amino acid sequence having at least 70% (preferably at least 80, 90, 95, 96, 97, 98 or 99%) sequence identity thereto, and in which the CDR sequences are as defined above.

Preferably, the antibody or fragment thereof for use according to the invention comprises:
(i) a light chain variable region comprising or consisting of the amino acid sequence set forth in SEQ ID NO: 9 or SEQ ID NO: 10, or an amino acid sequence having at least 70% (preferably at least 80, 90, 95, 96, 97, 98 or 99%) sequence identity thereto, and in which the CDR sequences VLCDR1-3 are as defined above; and
(ii) a heavy chain variable region comprising or consisting of the amino acid sequence set forth in SEQ ID NO: 11 or SEQ ID NO: 12, or an amino acid sequence having at least 70% (preferably at least 80, 90, 95, 96, 97, 98 or 99%) sequence identity thereto, and in which the CDR sequences are as defined above.

In a particular embodiment, the specific binding molecule for use according to the invention is a monoclonal antibody of the IgG1 isotype and comprises light chains of the K subtype. The L1M2 light chain is of the κ subtype and has the amino acid sequence set forth in SEQ ID NO: 13. The H4 heavy chain has the amino acid sequence set forth in SEQ ID NO: 14. In a particular embodiment, the specific binding molecule for use according to the invention is the L1M2H4 antibody that comprises the L1M2 light chain and the H4 heavy chain (which antibody is also referred to as MDX-124). Thus the specific binding molecule for use according to the invention may be a monoclonal antibody comprising or consisting of:
i) a light chain comprising or consisting of the amino acid sequence set forth in SEQ ID NO: 13, or an amino acid sequence having at least 70% (preferably at least 80, 90, 95, 96, 97, 98 or 99%) sequence identity thereto, and in which the CDR sequences VLCDR1-3 are as defined above; and
ii) a heavy chain comprising or consisting of the amino acid sequence set forth in SEQ ID NO: 14, or an amino acid sequence having at least 70% (preferably at least 80, 90, 95, 96, 97, 98 or 99%) sequence identity thereto, and in which the CDR sequences VHCDR1-3 are as defined above.

Similarly, the L2M2 light chain is of the κ subtype and has the amino acid sequence set forth in SEQ ID NO: 15. The H2 heavy chain has the amino acid sequence set forth in SEQ ID NO: 16. In a particular embodiment, the specific binding molecule for use according to the invention is the L2M2H2 antibody that comprises the L2M2 light chain and the H2heavy chain (which antibody is also referred to as MDX-222). Thus the specific binding molecule for use according to the invention may be a monoclonal antibody comprising:
i) a light chain comprising or consisting of the amino acid sequence set forth in SEQ ID NO: 15, or an amino acid sequence having at least 70% (preferably at least 80, 90, 95, 96, 97, 98 or 99%) sequence identity thereto, and in which the CDR sequences VLCDR1-3 are as defined above; and
ii) a heavy chain comprising or consisting of the amino acid sequence set forth in SEQ ID NO: 16, or an amino acid sequence having at least 70% (preferably at least 80, 90, 95, 96, 97, 98 or 99%) sequence identity thereto, and in which the CDR sequences VHCDR1-3 are as defined above.

In an alternative embodiment, the L1M2 light chain may be paired with the H2 heavy chain and the L2M2 light chain may be paired with the H4 heavy chain.

As is known to the skilled person, antibody chains are produced in nature with signal sequences. Antibody signal sequences are amino acid sequences located at the N-termini of the light and heavy chains, N-terminal to the variable regions. The signal sequences direct the antibody chains for export from the cell in which they are produced. If produced in a cellular expression system, the light and heavy chains with the amino acid sequences of SEQ ID NOs: 13-16 may be encoded with a signal sequence. The signal sequence of the L1M2 and L2M2 light chains is set forth in SEQ ID NO: 20; the signal sequence of the H2 and H4 heavy chains is set forth in SEQ ID NO: 21. If synthesised with a signal sequence, the L1M2 chain may thus be synthesised with the amino acid sequence set forth in SEQ ID NO: 22; the H4 chain may be synthesised with the amino acid sequence set forth in SEQ ID NO: 23; the L2M2 chain may be synthesised with the amino acid sequence set forth in SEQ ID NO: 24 and the H2 chain may be synthesised with the amino acid sequence set forth in SEQ ID NO: 25. Nucleotide sequences encoding such sequences may be easily derived by the skilled person, but examples of suitable nucleotide sequences which encode the antibody chains of SEQ ID NOs: 22-25, and may be used for their synthesis, are set forth in SEQ ID NOs: 26-29, respectively.

Sequence identity may be assessed by any convenient method. However, for determining the degree of sequence identity between sequences, computer programmes that make pairwise or multiple alignments of sequences are useful, for instance EMBOSS Needle or EMBOSS stretcher (both Rice, P. et al., Trends Genet. 16, (6) pp. 276-277, 2000) may be used for pairwise sequence alignments while Clustal Omega (Sievers F et al., Mol. Syst. Biol. 7:539, 2011) or MUSCLE (Edgar, R. C., Nucleic Acids Res. 32(5): 1792-1797, 2004) may be used for multiple sequence alignments, though any other appropriate programme may be used. Whether the alignment is pairwise or multiple, it must be performed globally (i.e. across the entirety of the reference sequence) rather than locally.

Sequence alignments and % identity calculations may be determined using for instance standard Clustal Omega parameters: matrix Gonnet, gap opening penalty 6, gap extension penalty 1. Alternatively, the standard EMBOSS Needle parameters may be used: matrix BLOSUM62, gap opening penalty 10, gap extension penalty 0.5. Any other suitable parameters may alternatively be used.

For the purposes of this application, where there is dispute between sequence identity values obtained by different methods, the value obtained by global pairwise alignment using EMBOSS Needle with default parameters shall be considered valid.

As set out above, the present invention provides a specific binding molecule (as defined above) for use in combination with various second active agents in both the treatment of cancer generally, and in the treatment of various specific cancers. Optionally additional active agents may be used such as third active agents as described hereinafter. In the alternative the treatments may be carried out without a third active agent, in particular with only the specific binding molecule and the second active agent. The specific binding molecule and the second active agent (and optionally third active agent, when present) act as active therapeutics. Any type of cancer may be treated according to the present invention, including carcinoma (including adenocarcinoma, squamous cell carcinoma, basal cell carcinoma, transitional cell carcinoma, etc.), sarcoma, leukaemia and lymphoma. Cancers that may be treated included melanoma, lung cancer, colorectal cancer, oesophageal cancer, stomach cancer, pancreatic cancer, breast cancer skin cancer, lymphoma (particularly Hodgkin lymphoma or mantle cell myeloma), bladder cancer, kidney cancer, mesothelioma, liver cancer and myeloma.

According to the invention, cancer of any stage (or grade) may be treated, including stage I, stage II, stage III and stage IV cancer. Both metastatic and localised (i.e. non-metastatic) cancer may be treated. In a preferred aspect, the cancer treated by the present invention is drug resistant, e.g. multidrug resistant (MDR). By drug resistant cancer is meant a cancer that is resistant to one chemotherapy drug. The drug to which the cancer is resistant may be the second or third active agent. MDR cancers are resistant to more than one chemotherapy drug, in particular more than one family of chemotherapy drug. MDR cancer may be resistant to 2, 3, 4 or 5 or more different chemotherapy drugs, or chemotherapy drug families (or classes). The term “MDR cancer” is well known in the art and is used in the present context in accordance with its meaning in the art. MDR cancer may be resistant to all known chemotherapy drugs. Multidrug resistance may be mediated by expression of one or more of the ABC transporters multidrug resistance protein 1 (MDR1), multidrug resistance-associated protein 1 (MRP1) and breast cancer resistance protein (BCRP). All three have broad substrate specificity and are able to expel chemotherapy agents of multiple different classes from cells that express them.

In a first aspect of the invention, provided herein is a specific binding molecule as defined above and a second active agent for use in the treatment of cancer in a subject, wherein the second active agent is a thymidylate synthetase inhibitor, a nucleobase analogue, a checkpoint inhibitor which blocks the interaction between PD-1 and PD-L1 or a proteasome inhibitor. That is to say, the invention provides a specific binding molecule as defined above in combination with a second active agent for the treatment of cancer, wherein the second active agent is a thymidylate synthetase inhibitor, a nucleobase analogue, a checkpoint inhibitor which blocks the interaction between PD-1 and PD-L1 or a proteasome inhibitor.

The nucleobase analogue may be any nucleobase analogue suitable for use in cancer therapy. As referred to herein a nucleobase analogue is a compound that can substitute for a natural nucleobase in a nucleic acid molecule, e.g. may form base pairs with the same partner base as the parent nucleobase to which it is an analogue. The nucleobase analogue may be any analogue of cytosine, guanine, adenine, thymine or uracil which has a cytotoxic effect on cancer cells, and/or is suitable for use in chemotherapy. In a particular embodiment the nucleobase analogue is a pyrimidine analogue, preferably a uracil analogue. 5-fluorouracil is a nucleobase analogue chemotherapeutic agent which may be used according to the invention.

A thymidylate synthetase inhibitor inhibits the enzyme thymidylate synthetase. Thymidylate synthetase catalyses conversion of deoxyuridine monophosphate (dUMP) to deoxythymidine monophosphate (dTMP), a nucleotide used in DNA synthesis. Inhibition of thymidylate synthetase thus inhibits dTMP production and DNA synthesis. A thymidylate synthetase inhibitor is thus any agent which inhibits the production of dTMP by thymidylate synthetase. Such an inhibitor may have any mode of action, e.g. competitive or non-competitive. Any thymidylate synthetase inhibitor suitable for use in chemotherapy can be used according to the invention. Several thymidylate synthetase inhibitors are known in the art (for instance the above-mentioned nucleobase analogue 5FU is a thymidylate synthetase inhibitor), and thymidylate synthetase inhibitors can also be identified using known techniques which measure thymidylate synthetase activity such as the tritiated 5-fluoro-dUMP binding assay (see e.g. Takezawa et al., British Journal of Cancer 103:354-361, 2010).

Most preferably, the nucleobase analogue or thymidylate synthetase inhibitor is 5-fluorouracil (5FU). The structure of 5FU is set forth in Formula I below:

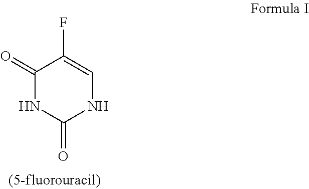

Another exemplary thymidylate synthetase inhibitor which may be used according to the invention is capecitabine, which is converted to 5FU in the body (i.e. is a 5FU pro-drug), and so has the same mechanism of action as 5FU. The structure of capecitabine is set forth in Formula II below:

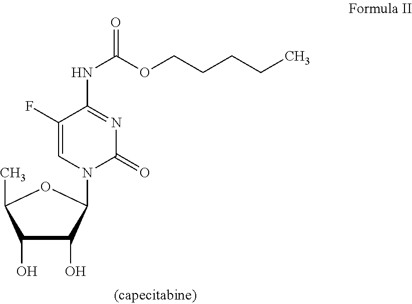

When the specific binding molecule is used in combination with a nucleobase analogue or thymidylate synthetase inhibitor (e.g. 5FU) the drugs may be used to treat any cancer. For instance the combination may be used to treat colorectal cancer, oesophageal cancer, stomach cancer, pancreatic cancer, breast cancer or skin cancer. In a preferred embodiment, the combination is used to treat pancreatic cancer or colorectal cancer. That is to say, in a preferred embodiment, the invention provides a specific binding molecule as defined above and 5FU for use in treatment of pancreatic cancer or colorectal cancer. In another preferred embodiment, the invention provides a specific binding molecule as defined above and capecitabine for use in the treatment of pancreatic cancer.

Thus, in one preferred embodiment the invention provides a specific binding molecule as defined above and 5FU for use in treatment of pancreatic cancer. In another preferred embodiment the invention provides a specific binding molecule as defined above and 5FU for use in treatment of colorectal cancer.

The pancreatic cancer treated according to this aspect of the invention may be any pancreatic cancer. In a particular embodiment the pancreatic cancer is pancreatic ductal adenocarcinoma.

As shown in the Examples below, when used to treat pancreatic cancer cell lines in vitro, the combination of the specific binding molecule for use according to the invention and 5FU demonstrate significant synergy in their anti-proliferative effect on the cell lines, demonstrating the unexpected benefit of combining these two drug types for cancer treatment.

Checkpoint inhibitors are molecules that block the activity of immune checkpoints. These inhibitors have found application as anti-cancer drugs by activating a patient's immune system to attack cancer cells. Immune checkpoints keep the immune system in check by preventing the killing of healthy cells and autoimmunity. They act as a “brake” on the immune system by preventing T-cell activation. Checkpoint proteins are expressed on the surface of immune cells and bind to checkpoint ligands on the surface of target cells or antigen-presenting cells, resulting in inhibition of immune cell activity.

PD-1 (programmed cell death protein 1) is an example of an immune checkpoint. PD-1 is expressed by T-cells and binds PD-L1 (programmed death ligand 1) and PD-L2 expressed on the surface of cells including target cells, lymphocytes and antigen-presenting cells. Activation of PD-1 by PD-L1 or PD-L2 binding inhibits T-cell activation and proliferation. Up-regulation of PD-L1 and/or PD-L2 by cancer cells thus acts as a protective mechanism to prevent their destruction by T-cells. Up-regulation of PD-L1 and/or PD-L2 by healthy cells in the vicinity of a tumour has a similar dampening effect on the immune response.

The present inventors have found that the combination of a specific binding molecule as defined herein and a checkpoint inhibitor which blocks the interaction of PD-1 and PD-L1 leads to unexpected advantageous effects in the treatment of cancer cells. A checkpoint inhibitor that blocks the interaction between PD-1 and PD-L1 may be any molecule which blocks that interaction, in order to inhibit PD-1 and block its activation, thus preventing the down-regulation of the immune response to the cancer. A checkpoint inhibitor which blocks the interaction between PD-1 and PD-L1 binds to one of these proteins and prevents interaction between the two proteins from taking place. Thus a checkpoint inhibitor which blocks the interaction between PD-1 and PD-L1 may bind to PD-1 or may bind to PD-L1. In preferred embodiments, the checkpoint inhibitor binds PD-1 or PD-L1. In particular, such a checkpoint inhibitor may bind to the PD-L1 binding site of PD-1, or the PD-1 binding site of PD-L1. It may be advantageous to use a checkpoint inhibitor which binds PD-1 to block the interaction between PD-1 and its ligands, in order to block interactions between PD-1 and both PD-L1 and PD-L2.

In particular embodiments of the invention, the checkpoint inhibitor which blocks the interaction between PD-1 and PD-L1 is an antibody (preferably a monoclonal antibody, or a derivative or fragment thereof) which binds PD-1. In other embodiments, the checkpoint inhibitor which blocks the interaction between PD-1 and PD-L1 is an antibody (preferably a monoclonal antibody, or a derivative or fragment thereof) which binds PD-L1. A number of such antibodies are known in the art, for instance Nivolumab (Bristol-Myers Squibb), a human monoclonal anti-PD1 IgG4 antibody; Pembrolizumab, a humanized IgG4 anti-PD-1 antibody (Merck); Cemiplimab (Regeneron/Sanofi), a human IgG4 anti-PD-1 antibody; Atezolizumab, a humanised anti-PD-L1 antibody (Genentech); and Durvalumab, a human anti-PD-L1 antibody (Medimmune/Astrazeneca), have all received regulatory approval and may be used according to the present invention. Many other such antibodies are currently in development/trials, such as Tislelizumab, a humanised anti-PD-1 antibody (BeiGene); and Avelumab, a fully human anti-PD-L1 antibody (Pfizer/Merck), and may also be used according to the present invention.

When the specific binding molecule is used in combination with a checkpoint inhibitor which blocks the interaction between PD-1 and PD-L1, the drugs may be used to treat any cancer. For instance, the combination may be used to treat melanoma, lung cancer, breast cancer, lymphoma (particularly Hodgkin lymphoma), stomach cancer, bladder cancer, oesophageal cancer, kidney cancer, mesothelioma, colorectal cancer or liver cancer. The combination may alternatively be used to treat any cancer with mismatch repair deficiency or microsatellite instability, and/or which is tumour mutational burden high (TMB-H).

Microsatellites (also known as “short tandem repeats”) are DNA sequences scattered throughout the genome (including both coding and non-coding regions) consisting of a repeating unit sequence. An individual microsatellite generally comprises between 10 and 60 copies of the repeating unit, which range from 1 to 6 base pairs in length. Due to the repeating nature of microsatellites, DNA polymerases are much more prone to making mistakes in these regions than in other regions of the genome. In cells with a functional mismatch repair (MMR) system, the MMR machinery “proofreads” newly-synthesised DNA strands, correcting errors made by the polymerase. Cancer cells which have a defect in the MMR machinery are unable to correct these errors, and thus have a 100 to 1000-fold increase in point mutations within their microsatellites. This increase in mutation rate in microsatellites is known as microsatellite instability (MSI) (Dudley et al., Clin Cancer Res 22(4): 813-820, 2016). A “microsatellite instability-high” (MSI-H) cancer is a cancer which demonstrates MSI. A “mismatch repair-deficient” cancer is a cancer lacking a functional MMR machinery.

A TMB-H cancer is defined as a tumour having ≥10 mutations/megabase. There is a substantial but not perfect correlation between TMB-H and MSI-H tumours, i.e. most but not all TMB-H tumours are also MSI-H, and vice versa. The cancer treated according to this embodiment of the invention may therefore be MSI-H but not TMB-H, TMB-H but not MSI-H, or MSI-H and TMB-H.

Preferably, the specific binding molecule as defined above is used in combination with a checkpoint inhibitor which blocks the interaction between PD-1 and PD-L1 to treat breast cancer or lung cancer. That is to say, in a preferred embodiment the invention provides a specific binding molecule as defined above and a checkpoint inhibitor which blocks the interaction between PD-1 and PD-L1 for use in the treatment of breast cancer. In another preferred embodiment, the invention provides a specific binding molecule as defined above and a checkpoint inhibitor which blocks the interaction between PD-1 and PD-L1 for use in the treatment of lung cancer.

The breast cancer treated according to this aspect of the invention may be any type of breast cancer, but in a particular embodiment the breast cancer is triple-negative breast cancer (i.e. breast cancer lacking expression of the oestrogen receptor, progesterone receptor and the hormone epidermal growth factor receptor HER2). Alternatively the breast cancer treated according to this aspect of the invention may be a hormone receptor-positive breast cancer, i.e. expressing one or more of the oestrogen receptor, progesterone receptor and HER2.

Similarly, the lung cancer treated according to this aspect of the invention may be any type of lung cancer, in particular it may be non-small cell lung cancer (NSCLC) or small cell lung cancer (SCLC).

As shown in the Examples below, when used to treat mouse models of lung and breast cancer, the combination of the specific binding molecule for use according to the invention and a checkpoint inhibitor which blocks the interaction between PD-1 and PD-L1 demonstrated significantly enhanced anti-tumour effects, demonstrating the unexpected benefit of combining these two drug types for cancer treatment.

The proteasome inhibitor may be any proteasome inhibitor suitable for use in cancer therapy. Proteasomes are protein complexes in the cell which degrade damaged (e.g. misfolded) or unneeded proteins by proteolysis after those proteins are tagged with ubiquitin. The predominant proteasome in mammals is the cytosolic 26S proteasome containing one 20S protein subunit (core particle) and two 19S regulatory cap subunits. The core particle consists of α subunits (structural) and β subunits (catalytic). Clinical and preclinical data supports a role for the proteasome in maintaining the immortal phenotype of myeloma cells. Proteasome inhibition is implicated in prevention of degradation of pro-apoptotic factors thereby triggering programmed cell death in neoplastic cells.

As referred to herein a proteasome inhibitor inhibits the activity of proteasomes partially or completely and has a cytotoxic effect on cancer cells, particularly multiple myeloma cancer cells. Preferred inhibitors inhibit the 26S proteasome by binding to its catalytic site but may exert their inhibition by any mode of action, e.g. competitive or non-competitive and the inhibition may be reversible or irreversible. Preferably the inhibitor inhibits the proteasome subunit β type-5 (PSMB5).

Preferred proteasome inhibitors are peptide analogues. Preferred inhibitors are bortezomib, ixazomib and carfilzomib.

The structures of bortezomib, ixazomib and carfilzomib are set forth in Formulae III-V below:

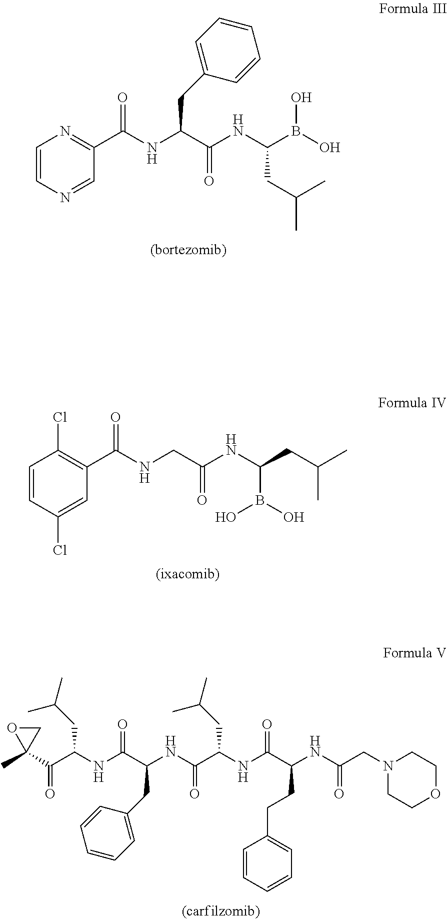

When the specific binding molecule is used in combination with a proteasome inhibitor (e.g. bortezomib, ixazomib or carfilzomib) the drugs may be used to treat any cancer. For instance, the combination may be used to treat melanoma, lung cancer, colorectal cancer, oesophageal cancer, stomach cancer, pancreatic cancer, breast cancer, skin cancer, lymphoma (particularly Hodgkin lymphoma or mantle cell myeloma), bladder cancer, kidney cancer, mesothelioma, liver cancer and myeloma. In a preferred embodiment, the combination is used to treat myeloma (also referred to as multiple myeloma) or mantle cell lymphoma. That is to say, in a preferred embodiment, the invention provides a specific binding molecule as defined above and bortezomib, ixazomib or carfilzomib for use in treatment of myeloma or mantle cell lymphoma (particularly bortezomib for use in treating myeloma).

Thus, in one preferred embodiment the invention provides a specific binding molecule as defined above and bortezomib, ixazomib or carfilzomib for use in treatment of myeloma or mantle cell lymphoma.

As shown in the Examples below, when used to treat myeloma cell lines in vitro, the combination of the specific binding molecule for use according to the invention and bortezomib demonstrate considerably improved anti-proliferative effects on the cell lines, demonstrating the unexpected benefit of combining these two drug types for cancer treatment. In particular the specific binding molecule was shown to potentiate the effect of the bortezomib.

In a further embodiment a third active agent may be used in the cancer treatment. The third active agent may be selected from the second active agents described herein for this or other embodiments of the invention (i.e. two second active agents may be used) or alternative therapeutic molecules may be used. In some aspects of the invention yet further active agents may be used, but in some aspects of the invention only said specific binding molecule and said second active agent (and optionally said third active agent) are used.

In a second aspect of the invention, provided herein is a specific binding molecule as defined above and a second active agent for use in the treatment of breast cancer in a subject, wherein the second active agent is selected from a taxane and a platinum-based chemotherapy agent. That is to say, the invention provides a specific binding molecule as defined above in combination with a second active agent for the treatment of breast cancer, wherein the second active agent is a taxane or a platinum-based chemotherapy agent.

The breast cancer treated according to this aspect of the invention may be any breast cancer. In one embodiment the breast cancer is triple-negative breast cancer. In another embodiment the breast cancer is a hormone receptor-positive breast cancer.

As set out above, in this aspect of the invention the specific binding molecule may be used in combination with a taxane. Functionally taxanes affect cell growth by binding to and stabilizing microtubules causing cell-cycle arrest and apoptosis. A taxane falls within the class of diterpenes and contains a taxadiene core. Any taxane which has a cytotoxic effect on cancer cells, and/or is suitable for use in chemotherapy may be used, for instance paclitaxel, docetaxel or cabazitaxel. In a preferred embodiment the taxane is paclitaxel. The structure of paclitaxel is set out in Formula VI below:

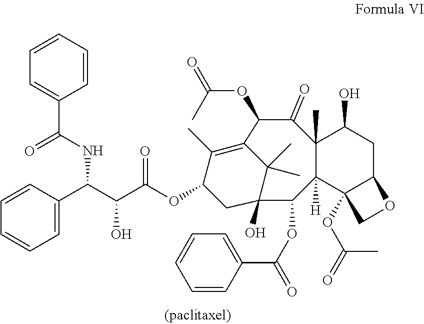 [SMILES: CC(=O)O[C@H]1C(=O)[C@@]2(C)[C@H]([C@H](OC(=O)c3ccccc3)[C@]3(O)C[C@H](OC(=O)[C@H](O)[C@@H](NC(=O)c4ccccc4)c4ccccc4)C(C)=C1C3(C)C)[C@]1(OC(C)=O)CO[C@@H]1C[C@@H]2O]

Paclitaxel may be provided in various formulations. For example, it may be provided in the form of paclitaxel protein-bound formulations, e.g. bound to albumin such as in nab-paclitaxel (an albumin-bound nanoparticle formulation of paclitaxel). Such alternative formulations of paclitaxel are considered encompassed by reference to paclitaxel. Similar considerations apply to other actives described herein.

As set out above, in this aspect of the invention the specific binding molecule may alternatively be used in combination with a platinum-based chemotherapy agent (i.e. a chemotherapy agent which contains a platinum ion or atom, particularly as a coordination complex of platinum). Platinum-based chemotherapy agents may be referred to as platinum-based antineoplastic agents, or platins. All platinum-based chemotherapy agents work in essentially the same way, by reacting with the N-7 position at guanine residues to form inter-and intrastrand DNA crosslinks and DNA-protein crosslinks. The crosslinks inhibit DNA synthesis and/or repair, and cause initiation of apoptosis (Shen et al., Pharmacol. Rev. 64:706-721, 2012). Any platinum-based chemotherapy agent may be used, for instance cisplatin, oxaliplatin, nedaplatin or carboplatin. In a preferred embodiment the platinum-based chemotherapy agent is cisplatin. The structure of cisplatin is set out in Formula VII below:

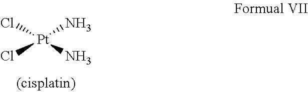

Thus in a preferred embodiment, the second aspect of the invention provides a specific binding molecule as defined above and a second active agent for use in the treatment of breast cancer in a subject, wherein the second active agent is selected from paclitaxel and cisplatin (that is to say, wherein the second active agent is paclitaxel or cisplatin). In a particular embodiment, the invention provides a specific binding molecule as defined above and paclitaxel for use in the treatment of breast cancer in a subject. In another embodiment, the invention provides a specific binding molecule as defined above and cisplatin for use in the treatment of breast cancer in a subject.

As shown in the Examples below, when used to treat a breast cancer cell line in vitro (specifically a triple-negative breast cancer cell line), the combination of the specific binding molecule for use according to the invention and cisplatin (or paclitaxel) demonstrate synergy in their anti-proliferative effect on the cell line, demonstrating the unexpected benefit of combining these two drug types for breast cancer treatment.

In a further embodiment a third active agent may be used in the breast cancer treatment. The third active agent may be selected from the second active agents described herein for this or other embodiments of the invention (i.e. two second active agents may be used) or alternative therapeutic molecules may be used. In some aspects of the invention yet further active agents may be used, but in some aspects of the invention only said specific binding molecule and said second active agent (and optionally said third active agent) are used.

In a third aspect of the invention, provided herein is a specific binding molecule as defined above and a second active agent for use in the treatment of pancreatic cancer in a subject, wherein the second active agent is a nucleoside analogue. That is to say, the invention provides a specific binding molecule as defined above in combination with a second active agent for the treatment of pancreatic cancer, wherein the second active agent is a nucleoside analogue.

As is known to the skilled person, nucleosides consist of a nucleobase conjugated to a 5-carbon sugar (ribose or 2′-deoxyribose). They differ to nucleotides in that nucleotides additionally comprise at least one phosphate group conjugated to the sugar moiety.

The nucleoside analogue may be an analogue of any nucleoside, i.e. an analogue of adenosine, deoxyadenosine, guanosine, deoxyguanosine, thymidine, uridine, cytidine or deoxycytidine. As referred to herein a nucleoside analogue is a compound that can substitute for a natural nucleoside in a nucleic acid molecule, e.g. may form base pairs with the same partner base as the parent nucleoside to which it is an analogue. The nucleoside analogue for use according to the invention, regardless of the natural nucleoside on which it is based, has a cytotoxic and/or chemotherapeutic effect, i.e. is suitable for use in cancer therapy. That is to say it is a chemotherapeutic nucleoside analogue. In a preferred embodiment the nucleoside analogue is an analogue of cytidine and/or deoxycytidine. Most preferably the nucleoside analogue is gemcitabine. The structure of gemcitabine is set out in Formula VIII below:

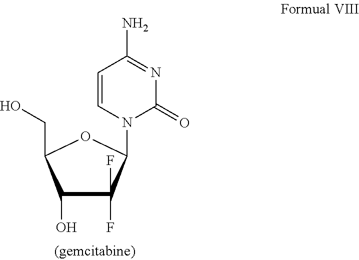

As shown in the Examples below, when used to treat a pancreatic cancer cell line in vitro, the combination of the specific binding molecule for use according to the invention and gemcitabine demonstrate significantly enhanced anti-proliferative effect on the cell line, demonstrating the unexpected benefit of combining these two drug types for pancreatic cancer treatment.

In a further embodiment a third active agent may be used in the pancreatic cancer treatment. The third active agent may be selected from the second active agents described herein for this or other embodiments of the invention (i.e. two second active agents may be used) or alternative therapeutic molecules may be used. In some aspects of the invention yet further active agents may be used, but in some aspects of the invention only said specific binding molecule and said second active agent (and optionally said third active agent) are used.

In a preferred aspect the third active agent is a taxane, preferably paclitaxel.

As shown in the Examples below, when used to treat pancreatic cancer in a mouse model, the combination of the specific binding molecule for use according to the invention, gemcitabine and paclitaxel demonstrated significantly enhanced anti-proliferative effect on the tumours, demonstrating the unexpected benefit of combining these drug types for pancreatic cancer treatment. Thus in a preferred aspect pancreatic cancer is to be treated using gemcitabine and paclitaxel.

Preferred combinations for cancer treatment are as set out in the examples.

In all of the aspects of the invention set out above, the cancer treated according to the invention may express ANXA1 (by which is meant that the cells in the cancer express ANXA1, e.g. on the cells' surface). It is straightforward for the skilled person to determine whether a cancer expresses ANXA1. ANXA1 expression may be analysed in a biopsy sample of a cancer, e.g. at the protein level by immunohistochemistry analysis of a sample. A sample may be immunostained using an anti-ANXA1 antibody (such as the antibodies described above) to detect ANXA1 expression, following standard procedures in the art. By permeabilizing a sample (e.g. using a detergent, as is standard in the art) both intracellular and extracellular ANXA1 may be detected.

Alternatively, ANXA1 expression may be analysed at the nucleic acid level, e.g. by quantitative PCR (qPCR). mRNA may be extracted from a tissue sample and reverse transcribed into DNA using procedures standard in the art. ANXA1 expression levels may then be determined by quantitative amplification of a target ANXA1 sequence. Suitable qPCR techniques, e.g. TaqMan, are well known in the art.

In a particular embodiment, the cancer overexpresses ANXA1. By “overexpresses ANXA1” is meant that the cancer expresses ANXA1 at a higher level than healthy tissue from the same source. That is to say, the cancerous cells express ANXA1 at a higher level than do healthy (i.e. non-cancerous) cells from the same source. By the same source is meant the same tissue. For instance, a pancreatic ductal adenocarcinoma may be considered to overexpress ANXA1 if it expresses ANXA1 at a higher level than does healthy pancreatic ductal tissue. Whether a cancer tissue overexpresses ANXA1 thus requires quantitative comparison of ANXA1 expression in at least two different tissues (the cancer tissue and a healthy control tissue). Any appropriate technique may be utilised to perform this comparison, though qPCR may be most suitable. It would be straightforward for the skilled person to determine whether a cancer overexpresses ANXA1. In a particular embodiment, the difference between the level of ANXA1 expression in the cancer that overexpresses it and healthy tissue is statistically significant. In other embodiments, ANXA1 expression is increased by at least 10, 20, 30, 40, 50, 60, 70, 80, 90 or 100% or more in the cancerous tissue relative to corresponding healthy tissue.

A cancer treated according to the present invention and which expresses ANXA1 may express ANXA1 on its surface (that is to say, ANXA1 may be expressed on the surface of the cells of the cancer). By expression of ANXA1 on the surface of cancer cells is meant that the cells express ANXA1, and the expressed ANXA1 is exported and localised on the cell surface. Cell surface expression of ANXA1 may be identified by immunohistochemistry, as described above. In particular, to analyse cell surface expression of ANXA1, the immunohistochemistry analysis is performed without cell permeabilization. This means that the antibody used to detect ANXA1 on the tissue is unable to enter the interior of the cells and only extracellular (e.g. surface-located) protein may be detected. Exported ANXA1 generally attaches to cell surfaces (rather than being released into plasma or any other extracellular space), and thus any ANXA1 detected by immunohistochemistry of non-permeabilized cells may be considered to be surface-located ANXA1. Nonetheless, following standard protocols, tissue may be washed prior to staining to remove loose extracellular material, including proteins.

One or more (preferably all) of the specific binding molecule, second and third active agents (when used) may be in free form (i.e. not bound to or associated with another molecule such as a carrier). Thus, in a preferred aspect no carrier is used for one or more (preferably all) of the specific binding molecule, second and third active agents.

In the alternative, one or more of the specific binding molecule, second and third active agents (when used) may be bound to, or associated with, a carrier. The carrier may be a particle, vesicle, or other solid support (e.g. a scaffold). In preferred aspects, a carrier, when used, is not a solid support, i.e. the specific binding molecule and/or second active agent (and/or third active agent when present) is associated with, but not bound to the carrier. In this aspect, the carrier may, for example, be used to package one or more of the specific binding molecule, second and third active agents without binding to those molecules. In one embodiment by way of example, the carrier may encapsulate one or more of the specific binding molecule, second and third active agents, e.g. the carrier may be a free-moving lipid vesicle, e.g. a liposome.

In another alternative, when a carrier is used that does bind to, or associate with the specific binding molecule, second and/or third active agents, the carrier that is used is proteinaceous.

Where a carrier is used it may bind to one or more of the specific binding molecule, second and/or third active agents. However, when used, the carrier preferably binds to only one of the specific binding molecule, second and/or third active agents. In that case different carriers may be used for the different molecules/agents and/or one or more of the molecules/agents may be in free form.

As described herein, the specific binding molecule and second active agents (and third active agents, when present) may be administered separately (e.g. in separate compositions), sequentially or simultaneously. In the latter case, the different molecules/agents may be provided in combination (i.e. in one composition). In all cases, and as discussed above, the molecules/agents may be provided for administration with or without a carrier. Where a carrier(s) is used, preferably only one of the specific binding molecule, second and/or third active agents is present on each carrier, i.e. when provided in the same composition, the other molecules/agents are in free form, or separate carriers may be used for the different molecules/agents. By way of example therefore, the specific binding molecule may be provided with a first carrier, and separately the second active agent may be provided with a second carrier and the third active agent (where present) may be provided in free form. However, in preferred aspects all of the agents are provided in free form.

The specific binding molecule, second active agent, and optionally the third (or further) active agent, when present, may each be administered to the subject to be treated in the form of a pharmaceutical composition. Such a composition may contain one or more pharmaceutically acceptable diluents, carriers or excipients. “Pharmaceutically acceptable” as used herein refers to ingredients that are compatible with other ingredients of the compositions as well as physiologically acceptable to the recipient. The nature of the composition and carriers or excipient materials, dosages etc. may be selected in routine manner according to choice and the desired route of administration, etc. Dosages may likewise be determined in routine manner and may depend upon the nature of the molecule, age of patient, mode of administration etc. As further discussed below, the specific binding molecule and second active agent (and optionally third active agent) may be administered to the subject in the same pharmaceutical composition or in separate pharmaceutical compositions.

A pharmaceutical composition may be prepared for administration to a subject by any suitable means. Such administration may be e.g. oral, rectal, nasal, topical, vaginal or parenteral. Oral administration as used herein includes buccal and sublingual administration. Topical administration as used herein includes transdermal administration. Parenteral administration as defined herein includes subcutaneous, intramuscular, intravenous, intraperitoneal and intradermal administration.

Pharmaceutical compositions as disclosed herein include liquid solutions or syrups, solid compositions such as powders, granules, tablets or capsules, creams, ointments and any other style of composition commonly used in the art. Suitable pharmaceutically acceptable diluents, carriers and excipients for use in such compositions are well known in the art. For instance, suitable excipients include lactose, maize starch or derivatives thereof, stearic acid or salts thereof, vegetable oils, waxes, fats and polyols. Suitable carriers or diluents include carboxymethylcellulose (CMC), methylcellulose, hydroxypropylmethylcellulose (HPMC), dextrose, trehalose, liposomes, polyvinyl alcohol, pharmaceutical grade starch, mannitol, lactose, magnesium stearate, sodium saccharin, talcum, cellulose, glucose, sucrose (and other sugars), magnesium carbonate, gelatin, oil, alcohol, detergents and emulsifiers such as the polysorbates. Stabilising agents, wetting agents, emulsifiers, sweeteners etc. may also be used.

Liquid pharmaceutical compositions, whether they be solutions, suspensions or other like form, may include one or more of the following: sterile diluents such as water for injection, Ringer's solution, isotonic sodium chloride, fixed oils such as synthetic mono-or diglycerides which may serve as a solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as EDTA; buffers such as acetates, citrates or phosphates and agents for the adjustment of tonicity such as dextrose. A parenteral preparation can be enclosed in ampoules, disposable syringes or multiple dose vials made of glass or plastic. An injectable pharmaceutical composition is preferably sterile.

Pharmaceutical compositions for use according to the present invention may be administered in any appropriate manner. The quantity and frequency of administration will be determined by such factors as the condition of the patient, and the type and severity of the patient's disease, although appropriate dosages may be determined by clinical trials. Conveniently a specific binding molecule and/or second active agent (and optionally a third active agent) for use according to the invention may be provided to a subject in a daily, weekly or monthly dose, or a dose in an intermediate frequency, e.g. a dose may be provided every 2, 3, 4, 5 or 6 days, every 2, 3, 4, 5 or 6 weeks, every 2, 3, 4, 5 or 6 months, annually or biannually. The dose may be provided for a total of at least 2 weeks, preferably at least 2 months, e.g. over a period of 3 to 24 months. The dose may be provided in the amount of from 100 ng/kg to 5 g/kg, e.g. 10 μg/kg to 1 g/kg body weight, for example from 1 mg/kg to 100 mg/kg. A dose is considered the application of a specific binding molecule or second active agent (or third active agent) at a single time or over a continuous time period, e.g. added as a single bolus or administered continuously over a discrete time period.

When a second (or third) active agent is used which is already licensed, the agent may conveniently be used at its licensed dose. For instance, 5FU may be administered as a 400 mg/m² intravenous bolus on day 1 followed by 2400-3000 mg/m² intravenously as a continuous infusion over 46 hours every 2 weeks. For a human adult of 70 kg this equates to around 11 mg/kg (for the bolus) to 68-85 mg/kg for each infusion (considered a single dose). Pembrolizumab may be administered as a 200 mg intravenous infusion every 3 weeks or a 400 mg intravenous infusion every 6 weeks (equating to around 6-11 mg/kg in an adult human). Bortezomib may be administered intravenously or subcutaneously twice weekly for at least 2 weeks at a dose of 1-5 mg for a human adult, followed by weekly doses in subsequent cycles. Ixazomib may be administered orally once weekly in a 4-week cycle at a dose of 1-5 mg for a human adult. Carfilzomib may be administered intraveneously twice weekly for three weeks in the first cycle at a dose of 10-100 mg for a human adult. Licensed doses of other existing therapies are well known in the art. Alternatively, the combination of the second active agent (and optionally third active agent) with the specific binding molecule against ANXA1 may enable the use of a lower dose of the second active agent (and/or optionally third active agent) than is currently licensed for use, particularly when synergy is observed between the two components. For example, the second active agent (and/or optionally third active agent) may be used at a dose which is up to 10, 20, 30, 40 or 50% or more lower than the existing licensed dose. The skilled clinician will be able to calculate an appropriate dose for a patient based on all relevant factors, e.g. age, height, weight, and condition to be treated.

The specific binding molecule and the second active ingredient may be provided in the amounts described above, e.g. as used conventionally or at reduced amounts. Conveniently the specific binding molecule and second active ingredient are used at a molar ratio from 2000:1 to 1:2000.

Preferably, the specific binding molecule and second active agent (or pharmaceutical composition(s) containing them) (and optionally third active agent) for use according to the invention are administered to the subject in need thereof in a therapeutically effective amount. By “therapeutically effective amount” is meant an amount sufficient to show benefit to the condition of the subject. Whether an amount is sufficient to show benefit to the condition of the subject may be determined by the physician/veterinarian.

The specific binding molecule as defined above and the second active agent (and optionally third active agent) may be administered to the subject separately, simultaneously or sequentially. “Separate” administration, as used herein, means that the specific binding molecule and the second active agent (and optionally third active agent) are administered to the subject at the same time, or at least substantially the same time, but by different administrative routes. “Simultaneous” administration, as used herein, means that the specific binding molecule and the second active agent (and optionally third active agent) are administered to the subject at the same time, or at least substantially the same time, by the same administrative route. By “sequential” administration, as used herein, is meant that the specific binding molecule and the second active agent (and optionally third active agent) are administered to the subject at different times. In particular, administration of the specific binding molecule is completed before administration of the second active agent (and optionally third active agent) commences (or vice versa). Sequential administration may be performed in which the two drugs are administered from 10 minutes to 30 days apart, e.g. from 1 hour to 96 hours (or 2 weeks) apart. When administered to a subject sequentially, the two drugs may be administered by the same administrative route or by different administrative routes.

The specific binding molecule for use according to the invention may also be administered to the subject in combination with radiotherapy and/or surgery.

As detailed above, the present invention is directed to the treatment of cancer in a subject. Treatment may be (or may be intended to be) curative, but may alternatively be palliative (i.e. designed merely to limit, relieve or improve the symptoms of the cancer, or to extend survival). Preferably the size of the tumour is reduced by the treatment or its rate of growth is stabilized or decreased. A reduction of at least 10%, preferably at least 20, 30 or 50% (e.g. up to 30, 50, 75 or 100%) in tumour size is preferred and the same levels of growth decrease are preferred.

The subject treated by the invention may be any mammal, e.g. a farm animal such as a cow, horse, sheep, pig or goat, a pet animal such as a rabbit, cat or dog, or a primate such as a monkey, chimpanzee, gorilla or human. Most preferably the subject is a human. The subject may be any animal (preferably human) who is suffering from cancer, or is suspected to be suffering from cancer. Thus the subject is an individual in need of treatment for cancer, or a specific cancer as set forth in the various aspects of the invention detailed above.

As detailed above, the first aspect of the invention provides a specific binding molecule which binds human ANXA1 as defined above and a second active agent (and optionally third active agent) for use in the treatment of cancer in a subject, wherein the second active agent is selected from a thymidylate synthetase inhibitor, a nucleobase analogue, a checkpoint inhibitor which blocks the interaction between PD-1 and PD-L1 and a proteasome inhibitor. This aspect of the invention can be seen as a method of treating cancer in a subject, comprising administering to the subject a specific binding molecule which binds human ANXA1 and a second active agent (and optionally third active agent), wherein the specific binding molecule is as defined above and the second active agent is selected from a thymidylate synthetase inhibitor, a nucleobase analogue, a checkpoint inhibitor which blocks the interaction between PD-1 and PD-L1 and a proteasome inhibitor. Such a method thus forms a fourth aspect of the invention. All features of this fourth aspect of the invention may be as defined above in respect of the first aspect.

Similarly, as set out above the second aspect of the invention provides a specific binding molecule which binds human ANXA1 and a second active agent (and optionally third active agent) for use in the treatment of breast cancer in a subject, wherein the specific binding molecule is as defined above, and the second active agent is selected from a taxane and a platinum-based chemotherapy agent. This aspect of the invention can alternatively be seen as providing a method of treating breast cancer in a subject, comprising administering to the subject a specific binding molecule which binds human ANXA1 and a second active agent (and optionally third active agent), wherein the specific binding molecule is as defined above in respect of the first aspect and the second active agent is selected from a taxane and a platinum-based chemotherapy agent. Such a method thus forms a fifth aspect of the invention. All features of this fifth aspect of the invention may be as defined above in respect of the second aspect.

Similarly, as set out above the third aspect of the invention provides a specific binding molecule which binds human ANXA1 and a second active agent (and optionally third active agent) for use in the treatment of pancreatic cancer in a subject, wherein the specific binding molecule is as defined above, and the second active agent is a nucleoside analogue. This aspect of the invention can alternatively be seen as providing a method of treating pancreatic cancer in a subject, comprising administering to the subject a specific binding molecule which binds human ANXA1 and a nucleoside analogue (and optionally third active agent), wherein the specific binding molecule is as defined above. Such a method thus forms a sixth aspect of the invention. All features of this sixth aspect of the invention may be as defined above in respect of the third aspect.

The first aspect of the invention may alternatively be seen as providing the use of a specific binding molecule which binds human ANXA1 in the manufacture of a medicament for treating cancer, wherein the specific binding molecule is as defined above, and said treatment of cancer comprises administering said medicament and a second active agent (and optionally third active agent) to a subject, wherein the second active agent is selected from a thymidylate synthetase inhibitor, a nucleobase analogue, a checkpoint inhibitor which blocks the interaction between PD-1 and PD-L1 and a proteasome inhibitor. Such a use thus forms a seventh aspect of the invention. All features of this seventh aspect of the invention may be as defined above in respect of the first aspect. In an alternative to this aspect the second active agent (and optionally third active agent) may be used to manufacture the medicament and the treatment comprises administration of the medicament and the specific binding molecule defined above.

The second aspect of the invention may alternatively be seen as providing the use of a specific binding molecule which binds human ANXA1 in the manufacture of a medicament for treating breast cancer, wherein the specific binding molecule is as defined above, and said treatment of breast cancer comprises administering said medicament and a second active agent (and optionally third active agent) to a subject, wherein the second active agent is selected from a taxane and a platinum-based chemotherapy agent. Such a use thus forms an eighth aspect of the invention. All features of this eighth aspect of the invention may be as defined above in respect of the second aspect. In an alternative to this aspect the second active agent (and optionally third active agent) may be used to manufacture the medicament and the treatment comprises administration of the medicament and the specific binding molecule defined above.

The third aspect of the invention may alternatively be seen as providing the use of a specific binding molecule which binds human ANXA1 in the manufacture of a medicament for treating pancreatic cancer, wherein the specific binding molecule is as defined above, and said treatment of pancreatic cancer comprises administering said medicament and a nucleoside analogue (and optionally third active agent) to the subject. Such a use thus forms a ninth aspect of the invention. All features of this ninth aspect of the invention may be as defined above in respect of the third aspect. In an alternative to this aspect the second active agent (and optionally third active agent) may be used to manufacture the medicament and the treatment comprises administration of the medicament and the specific binding molecule defined above.

In the seventh, eighth and ninth aspects of the invention, in line with the teaching above, the medicament which is made may comprise both the specific binding molecule which binds human ANXA1 and the second active agent (and optionally third active agent), or may comprise only the specific binding molecule which binds human ANXA1 or the second active agent (and optionally third active agent), in which case the two (or three) drugs are administered to the subject in the context of separate medicaments.

In a tenth aspect the invention provides a pharmaceutical composition comprising a specific binding molecule which binds human ANXA1 as described above, a second active agent (and optionally third active agent) as defined in the first aspect of the invention and one or more pharmaceutically-acceptable diluents, carriers or excipients. Pharmaceutical compositions and pharmaceutically-acceptable diluents, carriers or excipients are described above, all of which teaching is applicable to the pharmaceutical composition of the invention. The pharmaceutical composition of the invention may be used for the treatment of cancer, particularly cancers as described above in respect of the first aspect of the invention.

In an eleventh aspect the invention provides a kit comprising a specific binding molecule which binds human ANXA1, as defined above, and a second active agent (and optionally third active agent) as defined in respect of the first aspect of the invention. The specific binding molecule and second active agent (and optionally third active agent) may be provided as separate components, e.g. in separate compositions, which may be provided together in a single container or in separate containers. Alternatively, the specific binding molecule and second active agent (and optionally third active agent) may be provided in a single composition in a single container. Each therapeutic agent may be provided in any appropriate form, e.g. in an aqueous solution or as a lyophilisate.

In a twelfth aspect the invention provides a product comprising a specific binding molecule which binds human ANXA1 as defined and a second active agent (and optionally third active agent) for separate, simultaneous or sequential use in the treatment of cancer in a subject, wherein the second active agent is selected from a thymidylate synthetase inhibitor, a nucleobase analogue, a checkpoint inhibitor which blocks the interaction between PD-1 and PD-L1 and a proteasome inhibitor. The features of the product of the twelfth aspect and its use may be as defined above in respect of the first aspect.

In a thirteenth aspect the invention provides a product comprising a specific binding molecule which binds human ANXA1 as defined above and a second active agent (and optionally third active agent) for separate, simultaneous or sequential use in the treatment of breast cancer in a subject, wherein the second active agent is selected from a taxane and a platinum-based chemotherapy agent. The features of the product of the thirteenth aspect and its use may be as defined above in respect of the second aspect.

In a fourteenth aspect the invention provides a product comprising a specific binding molecule which binds human ANXA1 as defined above in respect of the first aspect and a nucleoside analogue (and optionally third active agent) for separate, simultaneous or sequential use in the treatment of pancreatic cancer in a subject. The features of the product of the fourteenth aspect and its use may be as defined above in respect of the third aspect.

In the products for use according to the invention the specific binding molecule and second active agent (and optionally third active agent) may be provided as separate components, e.g. in separate compositions, which may be provided together in a single container or in separate containers. Alternatively, the specific binding molecule and second active agent (and optionally third active agent) may be provided in a single composition in a single container. Each therapeutic agent may be provided in any appropriate form, e.g. in an aqueous solution or as a lyophilisate.

All documents cited in the present application are hereby wholly incorporated herein by reference.

The invention may be further understood by reference to the figures and non-limiting examples below:

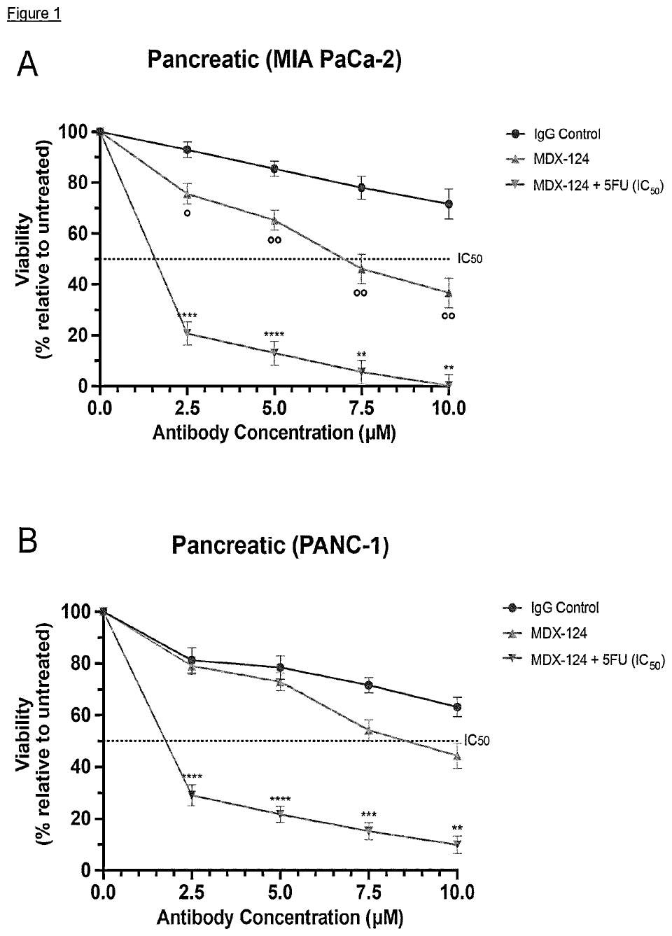

FIG. 1 shows that application of the antibody MDX-124 to the pancreatic cancer cell lines MIA PaCa-2 (A) and PANC-1 (B) significantly reduces cancer cell proliferation in-vitro both when the antibody is used as a single agent and when used in combination with chemotherapy using 5FU. The antibody was applied to the cells at a range of concentrations from 0 to 10 μM; 5FU was applied at its IC₅₀; ****p<0.0001, ***p<0.001 and **p<0.01 (MDX-124 v MDX-124+5FU IC₅₀) or ^{o}p<0.05 and ^{oo}p<0.01 (MDX-124 v IgG isotype control).

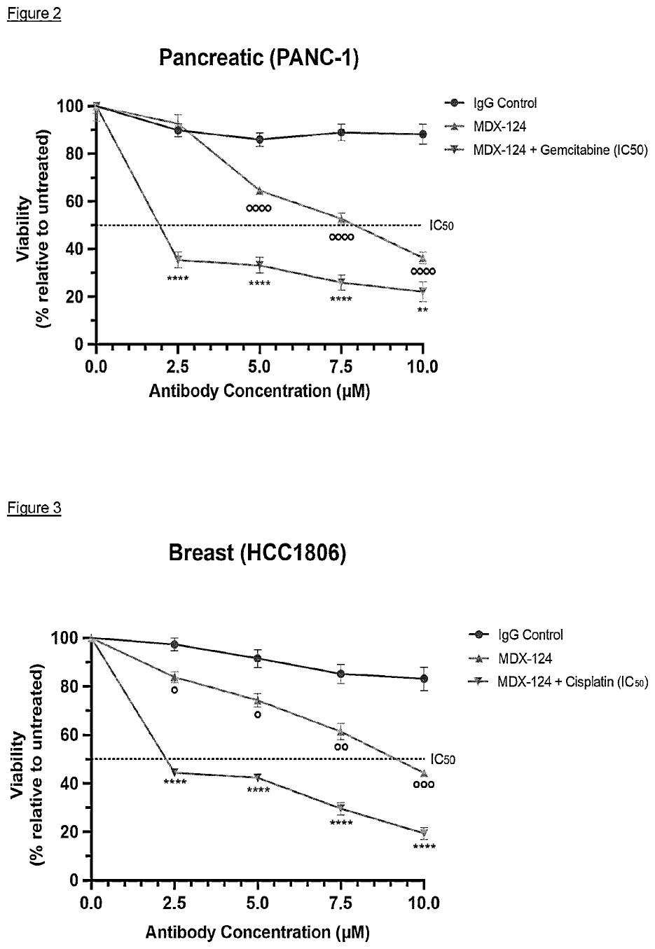

FIG. 2 shows that application of the antibody MDX-124 to the pancreatic cancer cell line PANC-1 significantly reduces cancer cell proliferation in-vitro both when the antibody is used as a single agent and when used in combination with chemotherapy using gemcitabine. The antibody was applied to the cells at a range of concentrations from 0 to 10 μM; gemcitabine was applied at its IC₅₀; ****p<0.0001, ***p<0.001 and **p<0.01 (MDX-124 v MDX-124+gemcitabine IC₅₀) or ^{oooo}p<0.0001 (MDX-124 v IgG isotype control).

FIG. 3 shows that application of the antibody MDX-124 to the breast cancer cell line HCC1806 significantly reduces cancer cell proliferation in-vitro both when the antibody is used as a single agent and when used in combination with chemotherapy using cisplatin. The antibody was applied to the cells at a range of concentrations from 0 to 10 μM; cisplatin was applied at its IC₅₀; ***p<0.0001 (MDX-124 v MDX-124+cisplatin IC₅₀) or ^{o}p<0.05, ^{oo}p<0.01 and ^{ooo}p<0.001 (MDX-124 v IgG isotype control). The figure is representative of two independent experiments.

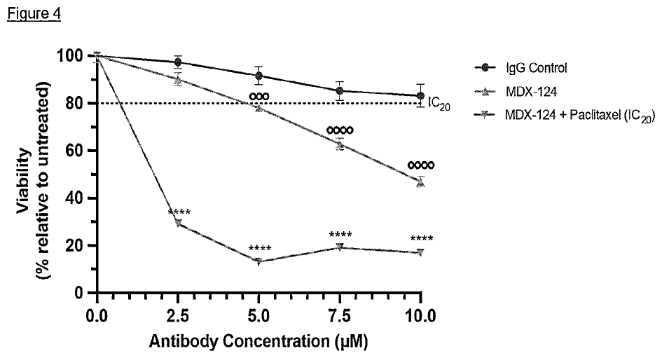

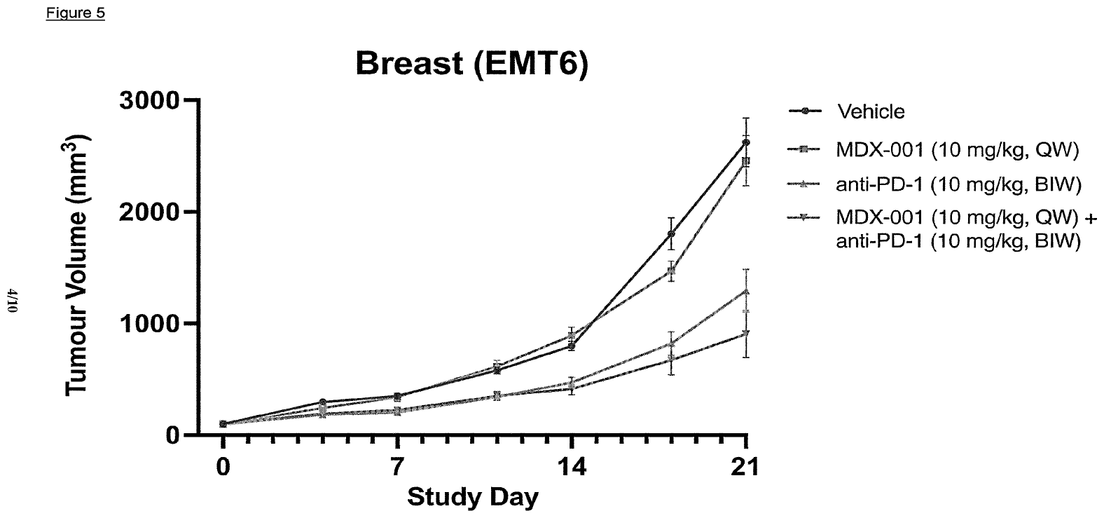

FIG. 4 shows that application of the antibody MDX-124 to the breast cancer cell line HCC1806 significantly reduces cancer cell proliferation in-vitro both when the antibody is used as a single agent and when used in combination with chemotherapy using paclitaxel. The antibody was applied to the cells at a range of concentrations from 0 to 10 μM; paclitaxel was applied at its IC₂₀; ****p<0.0001 (MDX-124 v MDX-124+paclitaxel IC₂₀) or ^{ooo}p<0.001 and ^{oooo}p<0.0001 (MDX-124 v IgG isotype control).

FIG. 5 shows mean tumour volumes in an EMT6 mouse model of breast cancer. Mice were inoculated with cancer cells and then administered either vehicle control (PBS), MDX-001 (10 mg/kg, QW), an anti-PD-1 antibody (10 mg/kg, BIW) or a combination of the MDX-001 and anti-PD-1 regimens (n=10 per group). Tumour volumes were calculated at the time points shown. As shown in the figure, the combination therapy group demonstrated the best results in terms of reducing tumour growth.

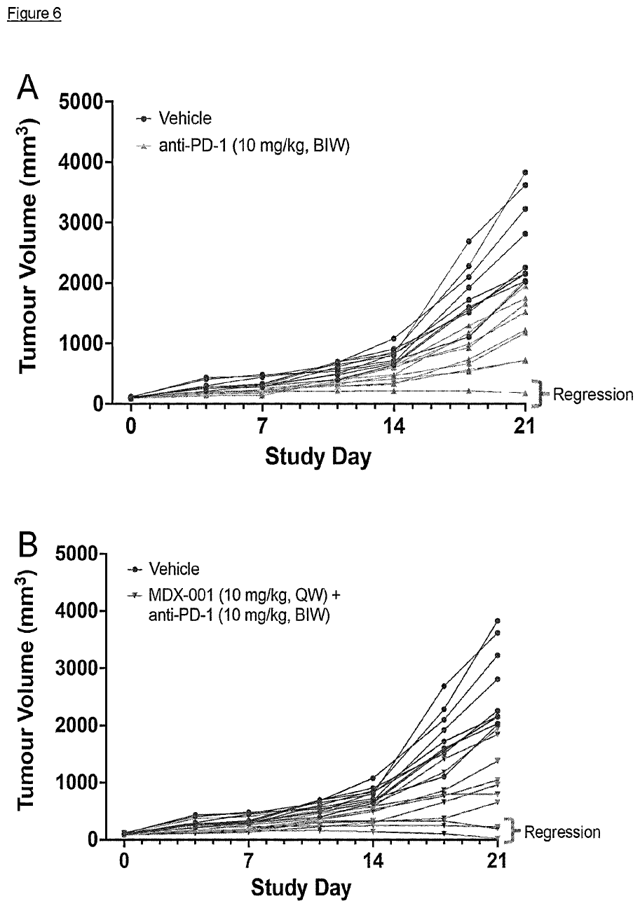

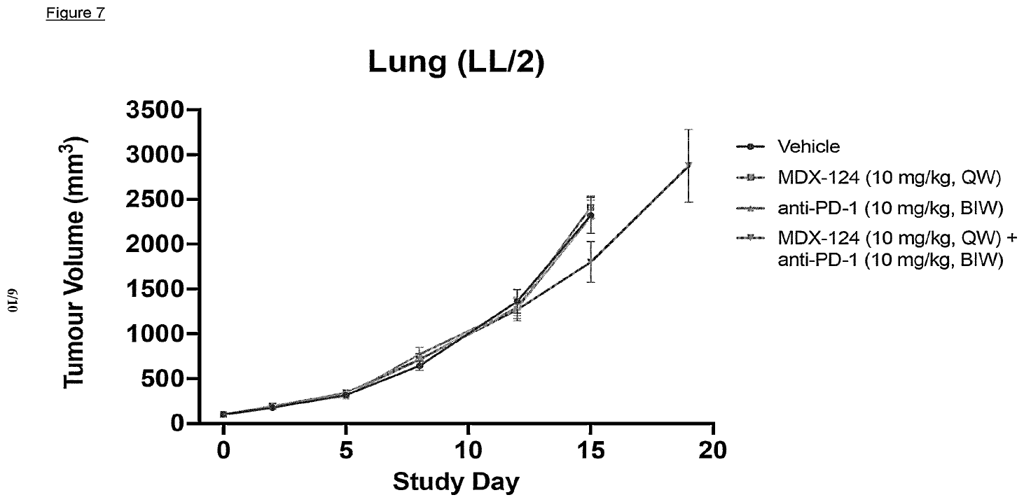

FIG. 6 shows the EMT6 tumour volumes of the individual mice that were analysed in FIG. 5. Results are shown comparing mice treated with vehicle with mice treated with anti-PD-1 antibody (A) or MDX-001 plus anti-PD-1 antibody combination therapy (B). As shown, more mice showed tumour regression in the group administered the combination of MDX-001 and anti-PD-1 antibody than in the group administered anti-PD-1 monotherapy.

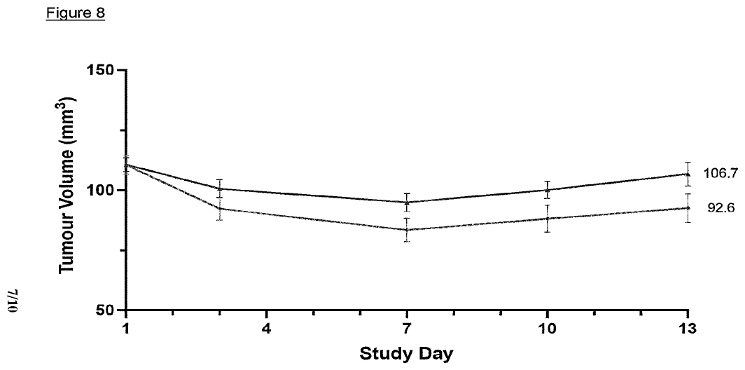

FIG. 7 shows mean tumour volumes in an LL/2 murine model of lung cancer. Mice were inoculated with cancer cells and then administered either vehicle control (PBS), MDX-001 (10 mg/kg, QW), an anti-PD-1 antibody (10 mg/kg, BIW) or a combination of the MDX-001 and anti-PD-1 regimens (n=10 per group). Tumour volumes were calculated at the time points shown. As shown in the figure, neither MDX-001 nor the anti-PD-1 antibodies displayed efficacy against the tumours when administered alone, but when administered in combination a significant anti-tumour effect was seen.

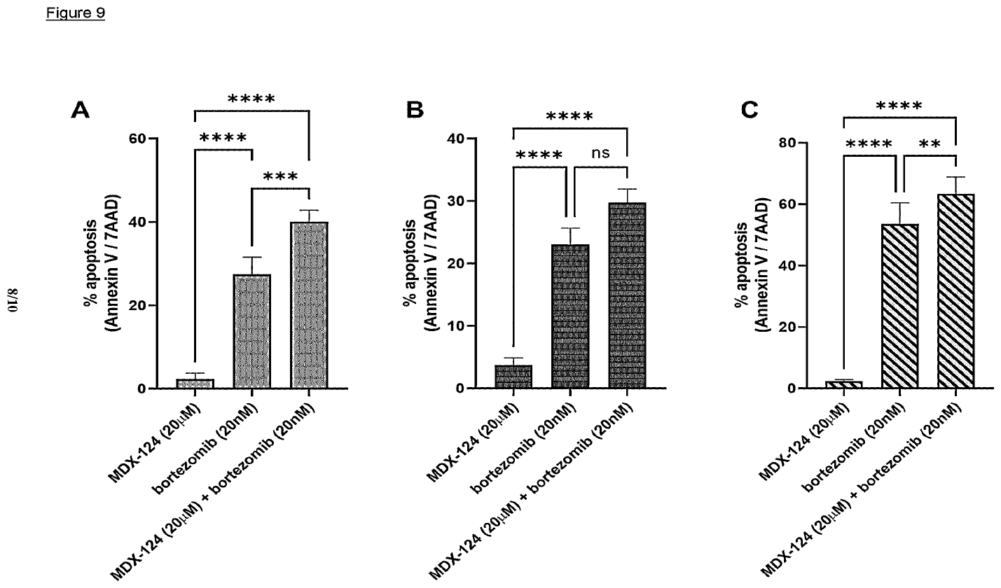

FIG. 8 shows mean tumour volumes in a Pan02 mouse model of pancreatic cancer. Mice were inoculated with cancer cells and then administered either gemcitabine (80 mg/kg, Q3D×4) and nab-paclitaxel (Abraxane, 30 mg/kg, Q3D×4) (n=50) or MDX-124 (10 mg/kg, twice weekly) plus gemcitabine (80 mg/kg, Q3D x4) and nab-paclitaxel (30 mg/kg, Q3D×4) (n=30). Tumour volumes were calculated at the time points shown and are shown as mean tumour volume±SEM.

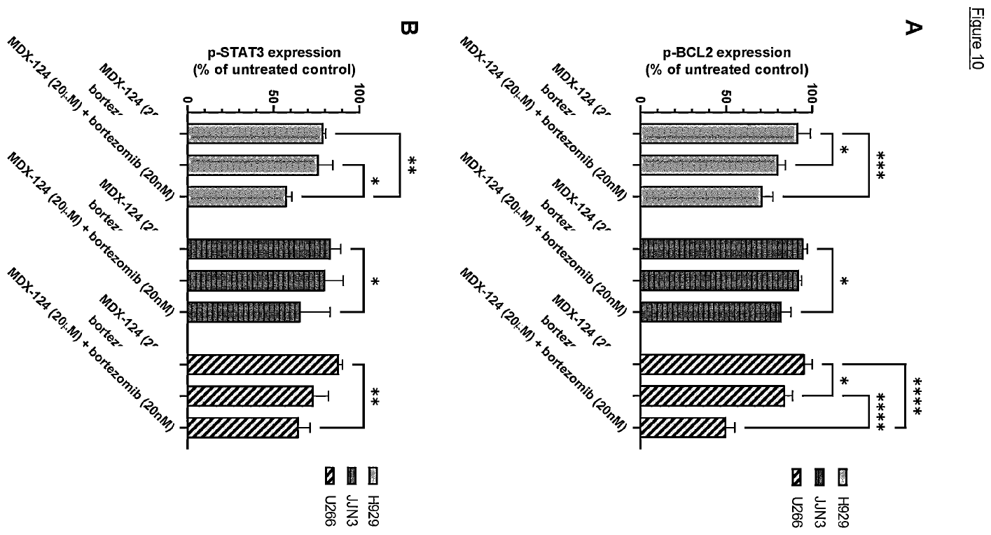

FIG. 9 shows the effects of MDX-124 +/−bortezomib on multiple myeloma cell line apoptosis. (A) H929 (B) JJN3 and (C) U266 human myeloma cell lines were treated with either MDX-124 (20 μM), bortezomib (20 nM) or a combination of both MDX-124 and bortezomib. All data are expressed as the mean±SD of three independent experiments, each carried out in duplicate. Statistical analysis was carried out using a 2-way ANOVA with Tukey's correction for multiple comparisons.

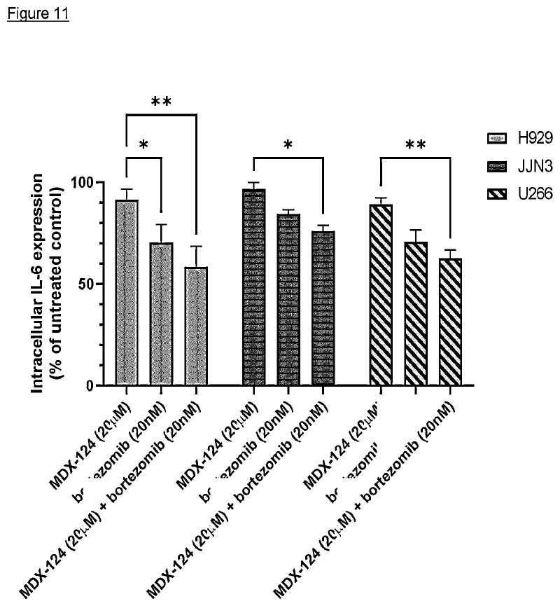

FIG. 10 shows the effect of MDX-124 and bortezomib on p-STAT3 and p-BCL2 expression in multiple myeloma cell lines. Aliquots of each human multiple myeloma cell line were treated with either MDX-124 (20 μM), bortezomib (20 nM) or a combination of MDX-124and bortezomib for 4h prior to staining with an Alexa488-labelled p-STAT3 (Tyr705) antibody and a PE-labelled p-BCL2 (pS70) antibody. Mean fluorescence intensity values for (A) p-BCL2 and (B) p-STAT3 after each respective treatment group were compared with untreated control cells. All data are expressed as the mean±SD of three independent experiments, each carried out in duplicate. Statistical analysis was carried out using a 2-way ANOVA with Tukey's correction for multiple comparisons.

FIG. 11 shows the effect of MDX-124 and bortezomib on intracellular IL-6 production in multiple myeloma cell lines. Aliquots of each human multiple myeloma cell line were treated with either MDX-124 (20 μM), bortezomib (20 nM) or a combination of MDX-124 and bortezomib for 24h prior to intracellular IL-6 analysis in fixed and permeabilised cells. Mean fluorescence intensity values for IL-6 for each respective treatment group were compared with untreated control cells. All data are expressed as the mean±SD of three independent experiments, each carried out in duplicate. Statistical analysis was carried out using a 2-way ANOVA with Tukey's correction for multiple comparisons.

### EXAMPLES

### Example 1—Testing of Anti-ANXA1 Antibody Combinations In Vitro Against Cancer Cell Lines

### Combination of MDX-124 and 5FU Against Pancreatic Cancer Cell Lines

An MTT cell proliferation assay was performed against the pancreatic cancer cell lines MIA-PaCa-2 and PANC-1. The cell lines were obtained from Public Health England Culture Collections. MIA-PaCa-2 is a human pancreatic carcinoma cell line; PANC-1 is a human pancreatic epithelioid carcinoma cell line. MIA PaCa-2 and PANC-1 cells were cultured in DMEM containing 10% FBS, 1% pen/strep and 1% L-glutamine at 37° C. in an atmosphere containing 5% CO₂.

MDX-124 is described above in the description: it is a humanised IgG1 antibody against ANXA1 with a light chain of SEQ ID NO: 13 and a heavy chain of SEQ ID NO: 14.

Cell proliferation was measured using the MTT colorimetric assay to measure cell metabolic activity. In the assay, NADPH-dependent cellular oxidoreductase enzymes reduce the yellow tetrazolium dye, MTT, to an insoluble purple formazan product, quantified by measuring absorbance at 500-600 nm using a spectrophotometer. The quantity of the formazan is proportional to the level of cell proliferation with rapidly dividing cells reducing a higher level of MTT. Assays were performed in triplicate. Cells were seeded in a final volume of 100 μl. MIA PaCa-2 and PANC-1 cells were seeded at a density of 1×10⁴ per well.

Cells were then cultured for 24 hr prior to assay, then cell proliferation was measured. In the proliferation assay, cells were cultured for 72 hours in the presence of an IgG isotype negative control (Thermo Fisher Scientific, USA, catalogue number 31154) at concentrations from 2.5-10 μM, in the presence of MDX-124 (2.5-10 μM) or in the presence of MDX-124 (2.5-10 μM) in combination with 5FU at either 100 μM(for MIA PaCa-2 cells) or 1 mM (for PANC-1 cells). The anti-proliferative effect of each treatment was measured as the percentage response compared to untreated control cells.

With both cell lines 5FU was used at its IC₅₀. The IC₅₀ concentration represents the concentration at which a substance exerts half of its maximal inhibitory effect. The IC₅₀ for each cell line was calculated by treating with a series of 10-fold dilutions of 5FU ranging from 1 nM to 10 mM. The MTT assay was used to calculate cancer cell viability after 72 hr incubation with 5FU at each concentration. This was repeated 8 times, with the mean concentration at which 50% of cells were not viable considered as the IC₅₀ value.

As expected, MDX-124 alone displayed a relatively potent anti-proliferative effect on both cell lines, particularly MIA-PaCa-2. Even so, when combined with 5FU (at its IC₅₀) cancer cell viability in respect of both cell lines was significantly reduced compared to either individual treatment (FIG. 1). Combination of MDX-124 with 5FU reduced cancer cell viability by 99.8% and 91.2% for MIA PaCa-2 and PANC-1 cells lines, respectively. The results were analysed by unpaired t-test and using ‘SynergyFinder’ software (lanevski et al., Nucleic Acids Research 48 (W1): W488-W493, 2020), which showed that MDX-124 has potent synergistic activity when used in combination with 5FU.

### Combination of MDX-124 and Gemcitabine Against Pancreatic Cancer Cell Line

An MTT cell proliferation assay was performed against the pancreatic cancer cell line PANC-1, as above.

Cell proliferation was measured using the MTT colorimetric assay as described above, using the same IgG isotype control. Cells were cultured in the presence of MDX-124 (2.5-10 μM) or in the presence of MDX-124 (2.5-10 μM) in combination with gemcitabine at 20 μM, the IC₅₀ for gemcitabine for PANC-1 cells. The IC₅₀ was calculated as described above, using a range of gemcitabine dilutions from 0.1 nM to 100 μM, with the assay being repeated three times to establish the mean concentration at which 50% of cells were not viable (i.e. the IC₅₀).

As expected, MDX-124 alone displayed a relatively potent anti-proliferative effect on the cell line. Even so, when combined with gemcitabine (at its IC₅₀) cancer cell viability was significantly reduced compared to either individual treatment (FIG. 2). The results were analysed by unpaired t-test.

### Combination of MDX-124 and Cisplatin Against Breast Cancer Cell Line

An MTT cell proliferation assay was performed against the triple-negative breast cancer cell line HCC 1806. The cell line was obtained from the ATCC. HCC1806 cells were cultured in DMEM containing 10% FBS, 1% pen/strep and 1% L-glutamine at 37° C. in an atmosphere containing 5% CO₂.

Cell proliferation was measured using the MTT colorimetric assay as described above, using the same IgG isotype control. Cells were cultured in the presence of MDX-124 (2.5-10 μM) or in the presence of MDX-124 (2.5-10 μM) in combination with cisplatin at 0.65 μM, the IC₅₀ for cisplatin for HCC1806 cells. The IC₅₀ was calculated as described above, using a range of cisplatin dilutions from 0.1 nM to 100 μM, with the assay being repeated four times to establish the mean concentration at which 50% of cells were not viable (i.e. the IC₅₀).

As expected, MDX-124 alone displayed an anti-proliferative effect on the cell line, which was substantially enhanced by combination with cisplatin (FIG. 3). The results were analysed by unpaired t-test and using ‘SynergyFinder’ software (supra) which showed that MDX-124 has potent synergistic activity when used in combination with cisplatin.

### Combination of MDX-124 and Paclitaxel Against Breast Cancer Cell Line

An MTT cell proliferation assay was performed against the triple-negative breast cancer cell line HCC1806.

Cell proliferation was measured using the MTT colorimetric assay as described above, using the same IgG isotype control. Cells were cultured in the presence of MDX-124 (2.5-10 μM) or in the presence of MDX-124 (2.5-10 μM) in combination with paclitaxel at 0.4 nM, the IC₂₀ for paclitaxel for HCC1806 cells. The IC₂₀ was calculated as described above, using a range of paclitaxel dilutions from 0 to 10 nM, with the assay being repeated twice to establish the mean concentration at which 20% of cells were not viable (i.e. the IC₂₀).

As expected, MDX-124 alone displayed an anti-proliferative effect on the cell line, which was substantially enhanced by combination with paclitaxel (FIG. 4). The results were analysed by unpaired t-test and using ‘SynergyFinder’ software (supra) which showed that MDX-124 has potent synergistic activity when used in combination with paclitaxel.

### Example 2—Testing of Anti-ANXA1 Antibody Combinations in In Vivo Models of Cancer

### Breast Cancer Model

Nine-week-old female BALB/c mice were inoculated subcutaneously with 5×10⁵ EMT6 triple-negative breast cancer cells. Once tumours reached 100 mm³ (as measured using calipers), mice (n=10 per group) were given either vehicle control (PBS), MDX-001 (10 mg/kg, weekly), anti-PD-1 antibody (10 mg/kg, twice weekly) or a combination regimen of MDX-001 (10 mg/kg, weekly) and anti-PD-1 antibody (10 mg/kg, twice weekly). Tumour volume was measured twice weekly for 3 weeks. The anti-PD-1 antibody used is the mouse antibody RMP-1-14 (Yamazaki et al., Journal of Immunology 175(3): 1586-1592, 2005); PBS was administered by intraperitoneal injection; antibodies were administered by intravenous injection.

As detailed above, MDX-001 is an anti-ANXA1 antibody, which is the parent of MDX-124. MDX-001 has light and heavy chains with the amino acid sequences set forth in SEQ ID NOs: 30 and 31, respectively.

Compared to the vehicle control, MDX-001 monotherapy did not significantly affect tumour growth, but tumour growth was significantly slower when mice were dosed with anti-PD-1 monotherapy. When the anti-PD-1 antibody and MDX-001 were used in combination, mean tumour volume was reduced by an additional 15% compared to anti-PD-1 monotherapy (FIG. 5). Notably, 30% of mice treated with MDX-001 plus anti-PD-1 combination therapy showed evidence of tumour regression at day 21 compared to day 18, versus only 10% receiving anti-PD-1 therapy alone (FIG. 6). No loss of body weight was seen in any of the treatment groups, and no adverse effects were seen with either MDX-001, anti-PD-1 or combination therapy treatment.

### Lung Cancer Model

Nine-week-old female C57BL/6 mice were inoculated subcutaneously with 3×10⁵ LL/2 lung cancer cells. Once tumours reached 100 mm³ (as measured using calipers), mice (n=10 per group) were given either vehicle control (PBS), MDX-124 (10 mg/kg, weekly), anti-PD-1 (10 mg/kg, twice weekly) or a combination regimen of MDX-124 (10 mg/kg, weekly) and anti-PD-1 (10 mg/kg, twice weekly). Drugs were administered as described above. Tumour volume was measured at days 2, 5, 8, 12 and 15, at which point the vehicle and monotherapy groups were terminated. The combination therapy group was measured again at day 19 and then terminated.

Results were analysed using a two-way repeated measures ANOVA/mixed-effects model. No significant differences were seen between the vehicle group and either MDX-124 or anti-PD-1 monotherapies. However, the MDX-124/anti-PD-1 combination group had significantly slower tumour growth than either vehicle control (P=0.022), MDX-124 alone (P=0.0003) or anti-PD-1 alone (P=0.037), demonstrating a synergistic effect for the combination of MDX-124 and anti-PD-1 therapy. The mice in the combination group had longer survival and remained on study until day 19, while the other groups were terminated on day 15 (FIG. 7). No body weight loss was detected in this study.

### Example 3—Testing of Anti-ANXA1 Antibody Combinations in an In Vivo Model of Pancreatic Cancer

### Pancreatic Cancer Model

Eight-week-old female C57BL/6 mice were inoculated subcutaneously with 5×10⁶ Pan02 pancreatic cancer cells and randomised to treatment groups once tumours reached 100 mm³ (as measured using calipers). During the initial 13-day treatment phase, mice were given either gemcitabine (Hospira Inc., Lake Forest, IL) (80 mg/kg, Q3D×4) and nab-paclitaxel (Abraxane, Celgene Corp., Summit, NJ; Celgene Europe, Germany) (30 mg/kg, Q3D×4) (n=50) or a combination regime of MDX-124 (10 mg/kg, twice weekly) plus gemcitabine (80 mg/kg, Q3D×4) and nab-paclitaxel (30 mg/kg, Q3D×4) (n=30). Drugs were administered as described above. A vehicle control was also performed with saline (BIW, 10mL/kg). Tumour volume was measured at days 3, 7, 10 and 13.

At day 13, mean tumour volume in the group receiving the combination regime of MDX-124 plus gemcitabine and nab-paclitaxel was 92.6 mm³, compared to a mean tumour volume of 106.7 mm³ in the group receiving gemcitabine and nab-paclitaxel alone (or relative to vehicle control treated mice, data not shown). Thus, the addition of MDX-124 to gemcitabine and nab-paclitaxel increased mean tumour growth inhibition compared to administration of gemcitabine and nab-paclitaxel alone in Pan02 mice (FIG. 8).

### Example 4—Testing of Anti-ANXA1 Antibody Combinations on Multiple Myeloma Cell Lines In Vitro

Several assays were used to assess the anti-cancer activity of MDX-124 in combination with bortezomib across a panel of human multiple myeloma cancer cell lines.

### Materials and Methods

### Apoptosis Assay

The effect of MDX-124, bortezomib and the combination of both agents on apoptosis was assessed using Annexin V and 7-AAD labelling. Human multiple myeloma cell lines (H929, JJN3 and U266, all from DSMZ, Leibniz Institute, German Collection of Microorganisms and Cell Cultures, GmbH, Germany) were incubated with either MDX-124 (20 μM), bortezomib (20 nM, Cell Signaling Technology, MA, USA) or the combination of both agents (fixed molar ratio of 1000:1) in 1 mL of RPMI media supplemented with 10% FCS for 48 h. The percentage of apoptosis above untreated control cell levels was quantified using flow cytometry for each respective treatment group.

### Expression of Apoptosis Related Proteins

The effects of MDX-124, bortezomib and the combination of both agents on the expression of 2 apoptosis related proteins, p-BCL2 and p-STAT3, was assessed using flow cytometry. Aliquots of each human multiple myeloma cell line were treated with either MDX-124 (20 μM), bortezomib (20 nM) or a combination of MDX-124 and bortezomib for 4 h prior to staining with an Alexa488-labelled p-STAT3 (Tyr705, from BD Biosciences, catalogue #557814) antibody and a PE-labelled p-BCL2 (pS70, BD Biosciences, catalogue #562532) antibody. Mean fluorescence intensity values for each treatment group were compared with untreated control cells.

### Expression of IL-6

The effects of each agent, both alone and in combination, on the expression of interleukin-6 (IL-6) were assessed using flow cytometry. Aliquots of each human multiple myeloma cell line were treated with either MDX-124 (20 uM), bortezomib (20 nM) or a combination of MDX-124 and bortezomib for 24h prior to intracellular IL-6 analysis in fixed and permeabilised cells. Mean fluorescence intensity values for IL-6 for each respective treatment group were compared with untreated control cells.

### Results

### Apoptosis Assay

MDX-124 alone had an impact on apoptosis, whilst bortezomib alone significantly induced apoptosis when compared to untreated control cells (FIG. 9). However, the addition of MDX-124 to bortezomib enhanced apoptosis in all cell lines tested when compared to bortezomib alone (FIG. 9).

### Expression of Apoptosis Related Proteins

STAT3 overexpression in multiple myeloma is associated with an adverse prognosis and is hypothesised to play a role in microenvironment-dependent treatment resistance. In addition to its pro-proliferative role, STAT3 upregulates anti-apoptotic proteins and leads to microRNA dysregulation in multiple myeloma (Chong et al., 2019, Cancers, Vol. 11(5), 731).

The BCL2 proteins are oncogenes that promote cell survival and are frequently upregulated in multiple myeloma, making them attractive targets (Gupta et al., 2021, Blood Lymphat. Cancer, Vol. 11, 11-24).

Both MDX-124 and bortezomib alone reduced the expression of p-BCL2 or p-STAT3 in the multiple myeloma cell lines tested (FIG. 10). However, the combination of MDX-124 and bortezomib caused a greater decrease in p-BCL2 and p-STAT3 when compared to either treatment alone in all cell lines tested (FIG. 10).

### Expression of IL-6

IL-6 is not only a growth factor, but also a survival factor in multiple myeloma, inhibiting apoptosis in myeloma cells. Reducing the effect of IL-6 has been linked to regression of tumour growth (Harmer et al., 2019, Front. Endocrinol., Vol. 9, doi: 10.3389/fendo.2018.00788).

Intracellular IL-6 expression was modestly reduced by both MDX-124 and bortezomib, however the combination of MDX-124 and bortezomib caused a greater reduction in all cell lines tested when compared to either treatment alone (FIG. 11).

Overall, these data suggest that in multiple myeloma cell lines the addition of MDX-124 to bortezomib potentiates the anti-cancer effects of either agent alone.

Sequence Listing
The sequences provided in the sequence listing are as shown
in the table follows:

| SEQ | Amino | |
|---|---|---|
| ID | acid or | |
| NO. | nucleotide | Sequence description |
| 1 | Amino acid | VLCDR1 of L1M2H4 and L2M2H2 |
| 2 | Amino acid | VLCDR2 of MDX-001, L1M2H4 and L2M2H2 |
| 3 | Amino acid | VLCDR3 of MDX-001, L1M2H4 and L2M2H2 |
| 4 | Amino acid | VHCDR1 of MDX-001, L1M2H4 and L2M2H2 |
| 5 | Amino acid | VHCDR2 of MDX-001, L1M2H4 and L2M2H2 |
| 6 | Amino acid | VHCDR3 of MDX-001, L1M2H4 and L2M2H2 |
| 7 | Amino acid | VLCDR1 of MDX-001 |
| 8 | Amino acid | VLCDR1 variant with S9T substitution |
| 9 | Amino acid | L1M2 light chain variable region |
| 10 | Amino acid | L2M2 light chain variable region |
| 11 | Amino acid | H4 heavy chain variable region |
| 12 | Amino acid | H2 heavy chain variable region |
| 13 | Amino acid | L1M2 light chain |
| 14 | Amino acid | H4 heavy chain |
| 15 | Amino acid | L2M2 light chain |
| 16 | Amino acid | H2 heavy chain |
| 17 | Amino acid | Human ANXA1 protein |
| 18 | Amino acid | Human ANXA1 fragment encoded by |
| | | ANXA1-004 |
| 19 | Amino acid | Human ANXA1 fragment encoded by |
| | | ANXA1-006 |
| 20 | Amino acid | Light chain signal sequence |
| 21 | Amino acid | Heavy chain signal sequence |
| 22 | Amino acid | L1M2 light chain pro sequence |
| 23 | Amino acid | H4 heavy chain pro sequence |
| 24 | Amino acid | L2M2 light chain pro sequence |
| 25 | Amino acid | H2 heavy chain pro sequence |
| 26 | Nucleotide | L1M2 light chain pro sequence |
| 27 | Nucleotide | H4 heavy chain pro sequence |
| 28 | Nucleotide | L2M2 light chain pro sequence |
| 29 | Nucleotide | H2 heavy chain pro sequence |
| 30 | Amino acid | MDX-001 light chain |
| 31 | Amino acid | MDX-001 heavy chain |

1. A method of treating cancer in a subject, comprising administering to the subject a specific binding molecule which binds human ANXA1 and a second active agent, wherein:
(i) the specific binding molecule comprises the complementarity-determining regions (CDRs) VLCDR1, VLCDR2, VLCDR3, VHCDR1, VHCDR2 and VHCDR3, each of said CDRs having an amino acid sequence as follows:
VLCDR1 has the sequence set forth in SEQ ID NO: 1, 7 or 8, or a modified version thereof comprising a conservative amino acid substitution at position 9 and/or 11;
VLCDR2 has the sequence set forth in SEQ ID NO: 2;
VLCDR3 has the sequence set forth in SEQ ID NO: 3;
VHCDR1 has the sequence set forth in SEQ ID NO: 4;
VHCDR2 has the sequence set forth in SEQ ID NO: 5; and
VHCDR3 has the sequence set forth in SEQ ID NO: 6; and
(ii) the second active agent is selected from a thymidylate synthetase inhibitor, a nucleobase analogue, a checkpoint inhibitor which blocks the interaction between PD-1 and PD-L1 and a proteasome inhibitor.

2. The method according to claim 1, wherein a third active agent is used in said treatment of cancer.

3. The method according to claim 1, wherein the second active agent is 5FU.

4. The method according to claim 3, wherein the cancer is pancreatic cancer or colorectal cancer.

5. The method according to claim 1, wherein the second active agent is an antibody which binds PD-1 or an antibody which binds PD-L1, optionally wherein the antibody which binds PD 1 is nivolumab, pembrolizumab, cemiplimab or tislelizumab, or the antibody which binds PD-L1 is atezolizumab, durvalumab or avelumab.

6. (canceled)

7. The method according to claim 5, wherein the cancer is breast cancer or lung cancer, wherein optionally the breast cancer is triple-negative breast cancer.

8. (canceled)

9. The method according to claim 1, wherein the second active agent is bortezomib, ixazomib or carfilzomib.

10. The method according to claim 9, wherein the cancer is myeloma or mantle cell lymphoma.

11. A method of treating breast cancer in a subject, comprising administering to the subject a specific binding molecule which binds human ANXA1 and a second active agent, wherein the specific binding molecule is as defined in claim 1, and the second active agent is selected from a taxane and a platinum-based chemotherapy agent;
preferably wherein the second active agent is selected from paclitaxel and cisplatin, optionally wherein a third active agent is used in said treatment of breast cancer.

12. (canceled)

13. A method of treating pancreatic cancer in a subject, comprising administering to the subject a specific binding molecule which binds human ANXA1 and a second active agent, wherein the specific binding molecule is as defined in claim 1, and the second active agent is a nucleoside analogue, preferably gemcitabine, optionally wherein a third active agent is used in said treatment of pancreatic cancer.

14. (canceled)

15. The method according to claim 13, wherein the second active agent is gemcitabine and the third active agent is paclitaxel.

16. The method according to claim 1, wherein the CDRs of the specific binding molecule have amino acid sequences as follows:
VLCDR1 has the sequence set forth in SEQ ID NO: 1;
VLCDR2 has the sequence set forth in SEQ ID NO: 2;
VLCDR3 has the sequence set forth in SEQ ID NO: 3;
VHCDR1 has the sequence set forth in SEQ ID NO: 4;
VHCDR2 has the sequence set forth in SEQ ID NO: 5; and
VHCDR3 has the sequence set forth in SEQ ID NO: 6.

17. The method according to claim 1 wherein the specific binding molecule is an antibody or fragment thereof, wherein optionally wherein the antibody or fragment thereof is humanised.

18. (canceled)

19. The method according to claim 17, wherein said antibody is a monoclonal antibody, or said antibody fragment is a Fab, Fab′ or F(ab′)₂ antibody fragment or an scFv molecule.

20. The method according to claim 19, wherein said antibody or fragment thereof comprises:
i) a light chain variable region comprising the amino acid sequence set forth in SEQ ID NO: 9 or 10, or an amino acid sequence having at least 70% sequence identity thereto; and
ii) a heavy chain variable region comprising the amino acid sequence set forth in SEQ ID NO: 11 or 12, or an amino acid sequence having at least 70% sequence identity thereto.

21-39. (canceled)

40. The method according to claim 20, wherein said specific binding molecule is a monoclonal antibody comprising:
i) a light chain comprising the amino acid sequence set forth in SEQ ID NO: 13, or an amino acid sequence having at least 70% sequence identity thereto; and
ii) a heavy chain comprising the amino acid sequence set forth in SEQ ID NO: 14, or an amino acid sequence having at least 70% sequence identity thereto.

41. The method according to claim 20, wherein said specific binding molecule is a monoclonal antibody comprising:
i) a light chain comprising the amino acid sequence set forth in SEQ ID NO: 15, or an amino acid sequence having at least 70% sequence identity thereto; and
ii) a heavy chain comprising the amino acid sequence set forth in SEQ ID NO: 16, or an amino acid sequence having at least 70% sequence identity thereto.

42. The method according to claim 1, wherein said cancer expresses ANXA1.

43. The method according to claim 1, wherein the specific binding molecule and second active agent, and optionally said third active agent when present, are administered to the subject separately, simultaneously or sequentially.

44. The method according to claim 1, wherein the subject is human.
